(19) United States
(12) Patent Application Publication
(10) Pub. No.: US 2015/0377889 A1
(43) Pub. Date: Dec. 31, 2015

(54) GALECTIN-3 AS A MARKER FOR PROSTATE CANCER

(71) Applicants: Avraham RAZ, West Bloomfield, MI (US); Elisabeth I. HEATH, Bloomfield Hills, MI (US); Balan VITALY, Amherst, NY (US); Lance K. HEILBRUN, (US)

(72) Inventors: Avraham Raz, West Bloomfield, MI (US); Elisabeth I Heath, Bloomfield Hills, MI (US); Vitaly Balan, Amherst, NY (US); Lance K Heilbrun, Beverly Hills, MI (US)

(73) Assignee: Wayne State University, Detroit, MI (US)

(21) Appl. No.: 14/768,094

(22) PCT Filed: Feb. 14, 2014
(86) PCT No.: PCT/US14/16561

§ 371 Date: Aug. 14, 2015

Related U.S. Application Data
(60) Provisional application No. 61/765,544, filed on Feb. 15, 2013.

(51) Int. Cl. G01N 33/574

(52) U.S. Cl. CPC G01N 33/57434; G01N 2333/96441; G01N 2333/4724

(57) ABSTRACT

Provided are methods and kits for the use of galectin-3, alone or in combination with prostate specific antigen (PSA), as a marker for the presence or absence of and/or activity of prostate cancer.

### CROSS REFERENCE TO RELATED APPLICATIONS

This application claims the benefit under 35 U.S.C. §119(e) to U.S. Provisional Patent Application No. 61/765,544 filed Feb. 15, 2013, the entire contents of which are incorporated by reference herein.

### STATEMENT REGARDING FEDERALLY SPONSORED RESEARCH

This invention was made with government support under Grant No. 2R37CA46120-19 awarded by the National Institutes of Health. The government has certain rights in the invention.

### FIELD OF THE DISCLOSURE

The present disclosure describes the use of galectin-3 as a marker for determining the presence or absence of prostate cancer and for assessing the effectiveness of a treatment. Galectin-3 can be used alone or in combination with the prostate specific antigen (PSA) marker to provide more reliable early detection methods for prostate cancer over the use of PSA alone.

### BACKGROUND OF THE DISCLOSURE

Prostate cancer, as its name indicates, is a cancer that develops in the prostate gland of the male reproductive system. Prostate cancer can be aggressive, in which cancer cells metastasize and move from the prostate gland to other parts of the body, such as the lymph nodes and the bones. It is the second leading cause of cancer-related death in men in the US, and its prevalence is increasing in developing countries.

When they emerge, symptoms associated with prostate cancer include pain, difficulty urinating, erectile dysfunction, and an inability to engage in sexual intercourse. Many men with prostate cancer, however, do not exhibit these symptoms early in the course of the disease and accordingly do not receive beneficial early treatment.

To date, the only blood biomarker that has been used to screen for the presence or absence of prostate cancer is prostate specific antigen (PSA) because it is often elevated in the serum of individuals with prostate cancer. However, PSA can also be elevated in individuals with other disorders of the prostate, or even in some cases in healthy individuals. For this reason, the U.S. Preventive Services Task Force in 2012 recommended against screening for the risk of having prostate cancer using the PSA test alone because the potential benefit of testing is outweighed by the expected harm of false diagnoses and over-treatment, which involve risk of complications. Currently, a prostate tissue biopsy is the only test that can fully confirm a diagnosis of prostate cancer.

### SUMMARY OF THE DISCLOSURE

Based on the foregoing, there is a need in the art for a more reliable test to detect the presence of, and/or monitor the progression of, prostate cancer. The present disclosure helps meet this need by providing methods of using galectin-3 (also known as Gal-3) as a marker for the presence or absence of prostate cancer, and monitoring the progression or regression of prostate cancer, either alone or complementary to PSA testing. Accordingly, the present disclosure describes methods for detecting the presence or absence of prostate cancer, determining the need for and/or response to treatment of prostate cancer, and/or monitoring the progression of prostate cancer disease, comprising the use of galectin-3 as a marker for prostate cancer either alone or with PSA or other markers. Some embodiments of the methods of the present disclosure comprise determining the level of cleaved and/or intact galectin-3. Other embodiments comprise determining the level of phosphorylated and/or unphosphorylated galectin-3.

An embodiment disclosed herein includes a method for determining presence or absence of prostate cancer in a subject comprising: determining a galectin-3 level in a biological sample obtained from the subject; comparing the determined galectin-3 level with a galectin-3 reference level; and determining prostate cancer is present in the subject if the level of galectin-3 in the biological sample is significantly elevated compared to the galectin-3 reference level. In one embodiment, the method further comprises determining a prostate specific antigen (PSA) level in a biological sample obtained from the subject; comparing the determined PSA level with a PSA reference level; and determining prostate cancer is present in the subject if (i) the level of galectin-3 in the biological sample is significantly elevated compared to the galectin-3 reference level and (ii) the level of PSA in the biological sample is significantly elevated compared to the PSA reference level.

Another embodiment disclosed herein includes a method for determining presence or absence of prostate cancer in a subject comprising: determining an intact galectin-3 level and a cleaved galectin-3 level in a biological sample obtained from the subject; comparing the determined levels of cleaved galectin-3 and intact galectin-3 in the biological sample; and determining prostate cancer is present in the subject if the level of intact galectin-3 is significantly elevated compared to the level of cleaved galectin-3. In one embodiment, the method further includes determining a PSA level in a biological sample obtained from the subject; comparing the determined PSA level with a PSA reference level; and determining prostate cancer is present in the subject if (i) the level of intact galectin-3 in the biological sample is significantly elevated compared to the level of cleaved galectin-3 and (ii) the level of PSA in the biological sample is significantly elevated compared to the PSA reference level. In another embodiment, the cleaved galectin-3 comprises the 1-107 fragment of galectin-3, the 108-250 fragment of galectin-3, or the 1-107 fragment of galectin-3 and the 108-250 fragment of galectin-3.

Another embodiment disclosed herein provides a method for determining the presence or absence of prostate cancer in a subject comprising: determining a phosphorylated galectin-3 level and an unphosphorylated galectin-3 level in a biological sample obtained from the subject; comparing the determined levels of phosphorylated galectin-3 and unphosphorylated galectin-3 in the biological sample; and determining prostate cancer is present in the subject if the level of phosphorylated galectin-3 is significantly elevated compared to the level of unphosphorylated galectin-3. In one embodiment, the method further comprises determining a PSA level in a biological sample obtained from the subject; comparing the determined PSA level with a PSA reference level; and determining prostate cancer is present in the subject if (i) the level of phosphorylated galectin-3 in the biological sample is significantly elevated compared to the level of unphosphorylated galectin-3 and (ii) the level of PSA in the biological sample is significantly elevated compared to the PSA reference level. In another embodiment, the galectin-3 is phosphorylated at Tyr-107 of galectin-3.

In another embodiment, the galectin-3 reference level, and/or the PSA reference level are individually derived from (i) an individual who does not have prostate cancer, (ii) a group of individuals who do not have prostate cancer; or (iii) the subject before diagnosis with prostate cancer. In yet another embodiment, the biological sample is a blood sample, a plasma sample, or a serum sample.

Another embodiment disclosed herein provides a method for determining the effectiveness of a treatment regimen for prostate cancer in a subject comprising: determining a galectin-3 level in a biological sample obtained from the subject; comparing the measured galectin-3 level with a galectin-3 reference level; and determining the treatment regimen is ineffective if the level of galectin-3 in the biological sample is significantly elevated or unchanged compared to the galectin-3 reference level. In one embodiment, the method further comprises determining a PSA level in a biological sample obtained from the subject; comparing the measured PSA level with a PSA reference level; and determining the treatment regimen is ineffective if (i) the level of galectin-3 in the biological sample is significantly elevated or unchanged compared to the galectin-3 reference level and (ii) the level of PSA in the biological sample is significantly elevated or unchanged compared to the PSA reference level.

Another embodiment disclosed herein provides a method for determining the effectiveness of a treatment regimen for prostate cancer in a subject comprising: determining a galectin-3 level in a biological sample obtained from the subject; comparing the measured galectin-3 level with a galectin-3 reference level; and determining the treatment regimen is effective if the level of galectin-3 in the biological sample is not significantly elevated or changed compared to the galectin-3 reference level. In one embodiment, the method further comprises determining a PSA level in a biological sample obtained from the subject; comparing the measured PSA level with a PSA reference level; and determining the treatment regimen is effective if (i) the level of galectin-3 in the biological sample is not significantly elevated or changed compared to the galectin-3 reference level and (ii) the level of PSA in the biological sample is not significantly elevated or changed compared to the PSA reference level.

Another embodiment disclosed herein provides a method for determining the effectiveness of a treatment regimen for prostate cancer in a subject comprising: determining an intact galectin-3 level and a cleaved galectin-3 level in a biological sample obtained from the subject; comparing the amounts of cleaved galectin-3 and intact galectin-3 in the biological sample; and determining the treatment regimen is ineffective if the level of intact galectin-3 in the biological sample is significantly elevated or unchanged compared to the level of cleaved galectin-3. In one embodiment, the method further comprises determining a PSA level in a biological sample obtained from the subject; comparing the measured PSA level with a PSA reference level; and determining the treatment regimen is ineffective if (i) the level of intact galectin-3 in the biological sample is significantly elevated or unchanged compared to the level of cleaved galectin-3 and (ii) the level of PSA in the biological sample is significantly elevated or unchanged compared to the PSA reference level.

Another embodiment disclosed herein provides a method for determining the effectiveness of a treatment regimen for prostate cancer in a subject comprising: determining an intact galectin-3 level and a cleaved galectin-3 level in a biological sample obtained from the subject; comparing the amounts of cleaved galectin-3 and intact galectin-3 in the biological sample; and determining the treatment regimen is effective if the level of intact galectin-3 in the biological sample is not significantly elevated or changed compared to the level of cleaved galectin-3. In one embodiment, the method further comprises determining a PSA level in a biological sample obtained from the subject; comparing the measured PSA level with a PSA reference level; and determining the treatment regimen is effective if (i) the level of intact galectin-3 in the biological sample is not significantly elevated or changed compared to the level of cleaved galectin-3 and (ii) the level of PSA in the biological sample is not significantly elevated or changed compared to the PSA reference level. In another embodiment, the cleaved galectin-3 comprises the 1-107 fragment of galectin-3, the 108-250 fragment of galectin-3, or the 1-107 fragment of galectin-3 and the 108-250 fragment of galectin-3.

Another embodiment disclosed herein provides a method for determining the effectiveness of a treatment regimen for prostate cancer in a subject comprising: determining a phosphorylated galectin-3 level and an unphosphorylated galectin-3 level in a biological sample obtained from the subject; comparing the amounts of phosphorylated galectin-3 and unphosphorylated galectin-3 in the biological sample; and determining the treatment regimen is ineffective if the level of phosphorylated galectin-3 in the biological sample is significantly elevated or unchanged compared to the level of unphosphorylated galectin-3. In one embodiment, the method further comprises determining a PSA level in a biological sample obtained from the subject; comparing the measured PSA level with a PSA reference level; and determining the treatment regimen is ineffective if (i) the level of phosphorylated galectin-3 in the biological sample is significantly elevated or unchanged compared to the level of unphosphorylated galectin-3 and (ii) the level of PSA in the biological sample is significantly elevated or unchanged compared to the PSA reference level.

Another embodiment disclosed herein provides a method for determining the effectiveness of a treatment regimen for prostate cancer in a subject comprising: determining a phosphorylated galectin-3 level and an unphosphorylated galectin-3 level in a biological sample obtained from the subject; comparing the amounts of phosphorylated galectin-3 and unphosphorylated galectin-3 in the biological sample; and determining the treatment regimen is effective if the level of phosphorylated galectin-3 in the biological sample is not significantly elevated or changed compared to the level of unphosphorylated galectin-3. In one embodiment, the method further comprises determining a PSA level in a biological sample obtained from the subject; comparing the measured PSA level with a PSA reference level; and determining the treatment regimen is effective if (i) the level of phosphorylated galectin-3 in the biological sample is not significantly elevated or changed compared to the level of unphosphorylated galectin-3 and (ii) the level of PSA in the biological sample is not significantly elevated or changed compared to the PSA reference level. In another embodiment, the galectin-3 is phosphorylated at position Tyr-107 of galectin-3.

In another embodiment, the galectin-3 reference level and/or PSA reference level are individually derived from (i) an individual who does not have prostate cancer, (ii) a group of individuals who do not have prostate cancer; (iii) the subject before diagnosis with prostate cancer; or (iv) the subject at the beginning of a treatment regimen for prostate cancer.

Another embodiment disclosed herein provides a method for monitoring the progression of prostate cancer in a subject comprising: determining a galectin-3 level in a biological sample obtained from the subject; comparing the measured galectin-3 level with a galectin-3 reference level; and determining that the prostate cancer has progressed if the level of galectin-3 in the biological sample is significantly elevated compared to the galectin-3 reference level. In one embodiment, the method further comprises determining a PSA level in a biological sample obtained from the subject; comparing the measured PSA level with a PSA reference level; and determining that the prostate cancer has progressed if (i) the level of galectin-3 in the biological sample is significantly elevated compared to the galectin-3 reference level and (ii) the level of PSA in the biological sample is significantly elevated compared to the PSA reference level.

Another embodiment disclosed herein provides a method for monitoring the regression of prostate cancer in a subject previously diagnosed with prostate cancer comprising: determining a galectin-3 level in a biological sample obtained from the subject; comparing the measured galectin-3 level with a galectin-3 reference level; and determining that the prostate cancer has regressed if the level of galectin-3 in the biological sample is not significantly elevated compared to the galectin-3 reference level. In one embodiment, the method further comprises determining a PSA level in a biological sample obtained from the subject; comparing the measured PSA level with a PSA reference level; and determining that the prostate cancer has regressed if (i) the level of galectin-3 in the biological sample is not significantly elevated compared to the galectin-3 reference level and (ii) the level of PSA in the biological sample is not significantly elevated compared to the PSA reference level.

Another embodiment disclosed herein provides a method for monitoring the progression of prostate cancer in a subject comprising: determining an intact galectin-3 level and a cleaved galectin-3 level in a biological sample obtained from the subject; comparing the amounts of cleaved galectin-3 and intact galectin-3 in the biological sample; and determining that the prostate cancer has progressed if the level of intact galectin-3 in the biological sample is significantly elevated compared to the level of cleaved galectin-3. In one embodiment, the method further comprises determining a PSA level in a biological sample obtained from the subject; comparing the measured PSA level with a PSA reference level; and determining that the prostate cancer has progressed if (i) the level of intact galectin-3 in the biological sample is significantly elevated compared to the level of cleaved galectin-3 and (ii) the level of PSA in the biological sample is significantly elevated compared to the PSA reference level.

Another embodiment disclosed herein provides a method for monitoring the regression of prostate cancer in a subject previously diagnosed with prostate cancer comprising: determining an intact galectin-3 level and a cleaved galectin-3 level in a biological sample obtained from the subject; comparing the amounts of cleaved galectin-3 and intact galectin-3 in the biological sample; and determining that the prostate cancer has regressed if the level of intact galectin-3 in the biological sample is not significantly elevated compared to the level of cleaved galectin-3. In one embodiment, the method further comprises determining a PSA level in a biological sample obtained from the subject; comparing the measured PSA level with a PSA reference level; and determining that the prostate cancer has regressed if (i) the level of intact galectin-3 in the biological sample is not significantly elevated compared to the level of cleaved galectin-3 and (ii) the level of PSA in the biological sample is not significantly elevated compared to the PSA reference level. In another embodiment, the cleaved galectin-3 comprises the 1-107 fragment of galectin-3, the 108-250 fragment of galectin-3, or the 1-107 fragment of galectin-3 and the 108-250 fragment of galectin-3.

Another embodiment disclosed herein provides a method for monitoring the progression of prostate cancer in a subject comprising: determining a phosphorylated galectin-3 level and an unphosphorylated galectin-3 level in a biological sample obtained from the subject; comparing the amounts of phosphorylated galectin-3 and unphosphorylated galectin-3 in the biological sample; and determining that the prostate cancer has progressed if the level of phosphorylated galectin-3 in the biological sample is significantly elevated compared to the level of unphosphorylated galectin-3. In one embodiment, the method further comprises determining a PSA level in a biological sample obtained from the subject; comparing the measured PSA level with a PSA reference level; and determining that the prostate cancer has progressed if (i) the level of phosphorylated galectin-3 in the biological sample is significantly elevated compared to the level of unphosphorylated galectin-3 and (ii) the level of PSA in the biological sample is significantly elevated compared to the PSA reference level.

Another embodiment disclosed herein provides a method for monitoring the regression of prostate cancer in a subject comprising: determining a phosphorylated galectin-3 level and an unphosphorylated galectin-3 level in a biological sample obtained from the subject; comparing the amounts of phosphorylated galectin-3 and unphosphorylated galectin-3 in the biological sample; and determining that the prostate cancer has regressed if the level of phosphorylated galectin-3 in the biological sample is not significantly elevated compared to the level of unphosphorylated galectin-3. In one embodiment, the method further comprises determining a PSA level in a biological sample obtained from the subject; comparing the measured PSA level with a PSA reference level; and determining that the prostate cancer has regressed if (i) the level of phosphorylated galectin-3 in the biological sample is not significantly elevated compared to the level of unphosphorylated galectin-3 and (ii) the level of PSA in the biological sample is not significantly elevated compared to the PSA reference level. In another embodiment, the galectin-3 is phosphorylated at position Tyr-107 of galectin-3.

In another embodiment, the galectin-3 reference level and/or PSA reference level are individually derived from (i) an individual who does not have prostate cancer, (ii) a group of individuals who do not have prostate cancer; (iii) the subject before diagnosis with prostate cancer; or (iv) the subject at the beginning of a treatment regimen for prostate cancer. In yet another embodiment, the biological sample is a blood sample, a plasma sample, or a serum sample.

Another embodiment disclosed herein provides a kit for determining the presence or absence of prostate cancer or the effectiveness or ineffectiveness of a treatment for prostate cancer or monitoring the progression or regression of prostate cancer in a subject using the method of any one of the preceding embodiments, wherein the kit comprises at least one galectin-3 detection assay. In another embodiment, the kit further comprises at least one PSA detection assay. In yet another embodiment, the kit comprises at least one intact galectin-3 detection assay and at least one cleaved galectin-3 detection assay.

In another embodiment, the kit comprises at least one intact galectin-3 detection assay, at least one cleaved galectin-3 detection assay, and at least one PSA detection assay. In yet another embodiment, the cleaved galectin-3 assay measures the 1-107 fragment of galectin-3, the 108-250 fragment of galectin-3, or the 1-107 fragment of galectin-3 and the 108-250 fragment of galectin-3.

In another embodiment, the kit comprises at least one phosphorylated galectin-3 detection assay and at least one unphosphorylated galectin-3 detection assay. In yet another embodiment, the kit comprises at least one phosphorylated galectin-3 detection assay, at least one unphosphorylated galectin-3 detection assay, and at least one PSA detection assay. In yet another embodiment, the assay measures phosphorylation at position Tyr-107 of galectin-3.

In another embodiment, the kit comprises all of at least one intact galectin-3 detection assay, at least one cleaved galectin-3 detection assay, at least one phosphorylated galectin-3 detection assay, and at least one unphosphorylated galectin-3 detection assay. In another embodiment, the kit further comprises at least one PSA detection assay.

In another embodiment, the detection assays can provide quantitative measurements of the detected cleaved galectin-3, detected intact galectin-3, detected phosphorylated galectin-3, detected unphosphorylated galectin-3, and/or detected PSA.

In yet another embodiment, the kit further includes instructions for performing the detection assays and instructions for determining whether the subject has prostate cancer.

In another embodiment, the kit further comprises reference samples for determining the reference levels of galectin-3 and/or PSA.

### BRIEF DESCRIPTION OF THE DRAWINGS

FIG. 1A-E demonstrate that phosphorylation of Tyr-107 on galectin-3 regulated its cleavage by PSA. In FIG. 1A, lane 1 is untreated galectin-3, lane 2 is PSA-treated galectin-3, and lane 3 is galectin-3 phosphorylated by c-Abl and treated with PSA. The top band of FIG. 1A shows immunoblotting using anti-galectin-3 antibody, and the bottom band shows immunoblotting using anti-Tyr(P) (pTyr) antibody. FIG. 1B shows tyrosine phosphorylation of galectin-3 in vivo blocked by c-Abl inhibitor. The top band of FIG. 1B shows immunoblotting using anti-phosphotyrosine antibody (pTyr), the middle shows anti-galectin-3 (HL31) (Gal-3), and the bottom shows overlapping Tyr(P) and galectin-3 (Mixed). FIG. 1C shows an SDS-PAGE gel demonstrating cleavage of galectin-3 with and without EGF treatment (+ and − lanes, respectively). FIG. 1D is a computer-generated image demonstrating the docking of galectin-3 Tyr(P)-107 on PTEN. Cys 124, Asp 92 and His 93 as labeled represent amino acids in the PTEN catalytic site. FIG. 1E shows the dephosphorylation of galectin-3 Tyr(P)-107 with PTEN. Lane 1 is wild type recombinant galectin-3 phosphorylated with c-Abl; lane 2 is phosphorylated galectin-3 treated with PTEN, and lane 3 is recombinant untreated galectin-3. The sample mixture was visualized with Coomassie Blue stain or immunoblotted with pTyr (pTyr blot), anti-galectin-3 (HL31) antibody (Gal-3 blot) or a mixture of the two (Gal-3+pTyr blot). The bottom panel of FIG. 1E depicts the normalized integrated intensity is shown, where AU is arbitrary units.

FIG. 2A-E. FIG. 2A demonstrates endothelial cell morphology resulting from co-cultures of BAMEC and LNCaP cells in the presence of recombinant full-length galectin-3 (Gal-3), fragment 1-107 (Gal-3 1-107) and fragment 108-250 (Gal-3 108-250). FIG. 2B shows a quantitative evaluation of the tube formation assay described herein. FIG. 2C shows results of the chemotaxis assay in LNCaP cells. Data points show the mean +/− S.E. (n=3) in each condition: Control, full-length galectin-3 (Gal-3), fragment 1-107 and fragment 108-250. FIG. 2D shows prostate cancer cell migration in the scratch assay, using LNCaP, Du145, and PC3M cell lines as labeled. FIG. 2E is a pathway graphic which describes possible mechanisms for the roles of PSA-resistant galectin-3 in the tumorigenesis and progression of prostate cancer.

FIG. 3 shows detection of galectin-3 in sera from a normal subject and a subject with prostate cancer.

FIG. 4 shows a standard curve for the ELISA assay of galectin-3 as described in Example 1. The x-axis is the concentration of galectin-3 (ng/ml), and the y-axis is the absorbance (OD450).

FIG. 5 shows the cleavage of galectin-3 in prostate tissue from healthy subjects vs. subjects with prostate cancer, and also shows PSA. FIGS. 5A-C and A′-C′ are from subjects with stage II prostate cancer. FIGS. 5A″-C″ are from healthy subjects. FIGS. 5A-A″ depict intact galectin-3. FIGS. 5A′-C′ depict cleaved galectin-3. FIGS. 5A″-C″ depict PSA.

### DETAILED DESCRIPTION

The present disclosure describes methods for detecting the presence or absence of prostate cancer, determining the need for and/or response to treatment of prostate cancer, and/or monitoring the progression of prostate cancer disease, comprising the use of galectin-3 as a marker for prostate cancer either alone or with prostate specific antigen (PSA). Some embodiments of the methods of the present disclosure comprise determining the level of cleaved and/or intact galectin-3. Other embodiments comprise determining the level of phosphorylated and/or unphosphorylated galectin-3. Also disclosed herein are kits for detecting the risk of having prostate cancer, determining the need for and/or response to treatment of prostate cancer, and/or monitoring the progression of prostate cancer disease, comprising the use of galectin-3 as a marker for prostate cancer either alone or with PSA.

“Determining” includes measuring, calculating, and/or analyzing a value or set of values associated with a sample by measurement of marker (i.e., analyte) levels in the sample. “Determining” may further comprise comparing levels against constituent levels in a sample or set of samples from the same subject or other subject(s). The galectin-3, PSA, and/or other biomarkers of the present disclosure can be determined by any of various conventional methods known in the art.

Galectin-3 and PSA are “biomarkers” or “markers” in the context of the present disclosure. Biomarkers include the protein forms of the markers as well as associated nucleic acids, oligonucleotides, and metabolites, together with their related metabolites, mutations, isoforms, variants, polymorphisms, modifications, fragments, subunits, degradation products, elements, and other analytes or sample-derived measures. Biomarkers can also include mutated proteins, mutated nucleic acids, variations in copy numbers, and/or transcript variants. Biomarkers also encompass combinations of any one or more of the foregoing measurements, including temporal trends and differences. Particular embodiments of biomarkers include the full length galectin-3 protein (SEQ ID NO:1); fragment 1-107 of the galectin-3 protein (SEQ ID NO:2); fragment 108-250 of the galectin-3 protein (SEQ ID NO:3); and PSA (SEQ ID NO:4).

Protein detection can comprise detection of full-length proteins, mature proteins, pre-proteins, polypeptides, isoforms, mutations, variants, post-translationally modified proteins, and variants thereof, and can be detected in any suitable manner. Levels of biomarkers can be determined at the protein level, e.g., by measuring the serum levels of proteins. Such methods are well-known in the art and include, e.g., immunoassays based on antibodies to proteins encoded by the genes, aptamers, or molecular imprints. Any biological material can be used for the detection/quantification of the protein or its activity. Alternatively, a suitable method can be selected to determine the activity of proteins. Such assays include, without limitation, protease assays, kinase assays, phosphatase assays, and reductase assays, among many others.

Using sequence information provided by public database entries for the biomarkers described herein, expression of the biomarker can be detected and measured using techniques well-known to those of skill in the art. For example, nucleic acid sequences in the sequence databases that correspond to nucleic acids of biomarkers can be used to construct primers and probes for detecting and/or measuring biomarker nucleic acids. These probes can be used in, e.g., Northern or Southern blot hybridization analyses, ribonuclease protection assays, and/or methods that quantitatively amplify specific nucleic acid sequences. As another example, sequences from sequence databases can be used to construct primers for specifically amplifying biomarker sequences in, e.g., amplification-based detection and quantitation methods such as reverse-transcription based polymerase chain reaction (RT-PCR) and PCR. When alterations in gene expression are associated with gene amplification, nucleotide deletions, polymorphisms, post-translational modifications and/or mutations, sequence comparisons in test and reference populations can be made by comparing relative amounts of the examined DNA sequences in the test and reference populations.

Variants of the sequences disclosed and referenced herein are also included. Variants of peptides can include those having one or more conservative amino acid substitutions. As used herein, a “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gin); Group 4: Arginine (Arg), Lysine (Lys), Histidine (His); Group 5: Isoleucine (Ile), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company which is incorporated by reference for its teachings regarding the same.

Variants of the protein and nucleic acid sequences disclosed or referenced herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to he protein and nucleic acid sequences disclosed or referenced herein and particularly including SEQ ID NO:1; SEQ ID NO.2; SEQ ID NO:3 and SEQ ID NO.4.

“% sequence identity” or “% identity” refers to a relationship between two or more sequences, as determined by comparing the sequences. In the art, “identity” also means the degree of sequence relatedness between proteins or nucleic acid sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992), each incorporated by reference herein for its teachings regarding the same. Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989), incorporated by reference herein for its teaching regarding the same) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990), incorporated by reference herein for its teaching regarding the same); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. incorporated by reference herein for its teaching regarding the same). Within the context of this disclosure it will be understood that where sequence analysis software is used for analysis, the results of the analysis are based on the “default values” of the program referenced. As used herein “default values” will mean any set of values or parameters which originally load with the software when first initialized.

A “dataset” as used herein is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored.

In certain embodiments of the present disclosure, a dataset of values is determined by measuring biomarkers from a healthy population, a Stage I prostate cancer population, a Stage II prostate cancer population, a Stage III prostate cancer population, or a Stage IV prostate cancer population. Datasets can be used by an interpretation function to derive a prostate cancer score, which can provide a quantitative measure of prostate cancer risk or presence in a subject.

In other embodiments, the amount of the biomarker(s) can be measured in a sample and used to derive a prostate cancer score, which prostate cancer score is then compared to a “reference level”. Reference levels can include “normal” or “control” levels or values, defined according to, e.g., discrimination limits or risk defining thresholds, in order to define cut-off points and/or abnormal values for prostate cancer. The reference level then is the level of one or more biomarkers or combined biomarker indices typically found in a subject who is not suffering from prostate cancer. Other terms for “reference levels” include “index,” “baseline,” “standard,” “healthy,” “pre-disease,” etc. Such normal levels can vary, based on whether a biomarker is used alone or in a formula combined with other biomarkers to output a score. Alternatively, the reference level can be a database of biomarker patterns from previously tested subjects who did not develop prostate cancer over a clinically relevant time period. Reference levels can also be derived from, e.g., a control subject or population whose prostate cancer diagnosis is known. In some embodiments, the reference value can be derived from one or more subjects who have been exposed to treatment for prostate cancer, or from subjects who have shown improvements in prostate cancer as a result of exposure to treatment. In some embodiments the reference level can be derived from one or more subjects who have not been exposed to treatment. A reference level can also be derived from disease activity algorithms or computed indices from population studies.

In further embodiments, “reference level” can refer to a standardized value for galectin-3 or PSA which represents a level not associated with any disease; a level associated with a particular stage of the disease (i.e., stage I, II, III or IV); or a level associated with a particular subject at the time of diagnosis, at the beginning of treatment, or at a time point during a treatment. The reference level can be a universal reference level which is useful across a variety of testing locations or can be a reference level specific for the testing location and specific immunoassay used to measure the galectin-3 or the PSA. In certain embodiments, the reference level, galectin-3 reference level, PSA reference level, and/or reference weighted score is derived from (i) an individual who does not have prostate cancer; (ii) a group of individuals who do not have prostate cancer; (iii) a subject before diagnosis of prostate cancer; or (iv) a subject at the time of diagnosis, at the beginning of a treatment regimen for prostate cancer or at particular time points during a treatment. Reference levels for a subject can also be related to time points of a subject not undergoing treatments to monitor the natural progression or regression of the disease.

“Interpretation functions,” as used herein, means the transformation of a set of observed data into a meaningful determination of particular interest; e.g., an interpretation function may be a predictive model that is created by utilizing one or more statistical algorithms to transform a dataset of observed biomarker data into a meaningful determination of prostate cancer disease or activity in a subject.

Galectin-3 is primarily a cytosolic protein that often can be found in the nucleus and is also secreted outside of the cell, despite the fact that it lacks the classical localization signals at the N-terminus. Galectin-3 is also secreted into the extracellular matrix (ECM), where it binds to the ECM proteins laminin, fibronectin and collagen IV. Galectin-3 is involved with a variety of extracellular functions, such as cell adhesion, migration, invasion, angiogenesis, immune functions and inflammatory responses, apoptosis and endocytosis, as well as in cell proliferation, cell motility, and differentiation. Galectin-3 can be found in a wide variety of tissues, including blood, and is expressed in different cell lineages at different developmental and pathological stages. Most importantly, galectin-3 is expressed in a wide range of tumor cells and its expression is associated with cell growth, tumorigenesis, angiogenesis, apoptosis resistance, tumor progression, adhesion, differentiation, inflammation, transformation, invasion, and metastasis.

The functions of galectin-3 are dependent on its localization and post-translational modifications, such as phosphorylation and cleavage, and on its multivalency. Galectin-3 is an approximately 30 kDa carbohydrate-binding protein belonging to the galectin gene family. It is composed of three distinct structural motifs: an N-terminal domain consisting of twelve amino acid residues including a serine phosphorylation site, followed by an N-terminal collagen-like sequence containing Pro-Gly-Tyr tandem repeats, and a carbohydrate-binding C-terminal domain. Galectin-3 is the only member of the galectin family that exhibits multivalency; i.e., it can form homodimers and homopentamers through intermolecular interactions involving its N-terminal domain. This oligomerization allows the functions of galectin-3 described above by forming ordered galectin-glycan structures on the cell surface, or through direct engagement of specific cell surface glycoconjugates by traditional ligand-receptor binding.

Prior to the present disclosure, the association of galectin-3 levels with a risk of having prostate cancer was controversial. Indeed, previous work demonstrated that expression of galectin-3 was significantly decreased in subjects with prostate cancer when compared to normal and premalignant tissue. Further, it was also previously demonstrated that the amount of certain types of cleaved galectin-3 increased during the malignant transformation and progression of human cancer. This is because galectin-3 can be cleaved by matrix metalloproteinases (MMP)-2 and MMP-9 between Ala-62 and -63, as well as by PSA at tyrosine 107 (Tyr-107). As both MMPs and PSA are present in prostate cancer tissue, most extracellular galectin-3 is cleaved either after alanine 62 or Tyr-107.

Tyrosines at positions 79, 107 and 118 of the galectin-3 polypeptide can be phosphorylated in vitro and in vivo by c-Abl kinase, and Tyr-107 is the main target of c-Abl phosphorylation. Active PSA cleaves galectin-3 between the amino acids Tyr-107 and glycine 108, which results in a loss of its multivalency.

In some cases, however, intact galectin-3 is observed in the presence of PSA. Substitution of proline at position 64 for histidine (the H64P substitution) in galectin-3 prevents cleavage by MMPs, and phosphorylation of Tyr-107 prevents cleavage by PSA. A high level of uncleaved galectin-3 in the presence of MMPs and PSA in prostate tissue can be explained either by the presence of the H64P substitution, or phosphorylation of Tyr-107.

The tumor suppressor protein phosphatase and tensin homolog (PTEN) dephosphorylates galectin-3. Almost 70% of all prostate cancer patients have lost one or both copies of the PTEN gene, and its activity is thus downregulated in these patients. A decrease in PTEN activity, as is seen in most prostate cancer patients, should result in reduced dephosphorylation (i.e., greater phosphorylation) of galectin-3 in prostate cancer patients compared to normal. This higher level of phosphorylated galectin-3 in turn would result in less cleavage of the protein, greater retained multivalency of the protein, and thus greater activity of the tumorigenic activity of the protein.

It is important to note that galectin-3 cleaved by PSA cannot create oligomers through its collagen-like sequence, while cleavage of galectin-3 by MMPs still permits this oligomerization. The ability of galectin-3 to create its characteristic lattice and thus induce cell-cell interactions is dependent on this oligomerization. Therefore, cleavage of galectin-3 by MMPs permits oligomerization and the tumorigenic effects of galectin-3 can proceed, whereas cleavage by PSA destroys galectin-3 ability to oligomerize and adversely affects its tumorigenic capacity. Thus, an increase in intact galectin-3 levels and an increase in the levels of phosphorylated galectin-3 are associated with an increased risk of having prostate cancer.

In Example 1, galectin-3 levels in the blood of prostate cancer patients were analyzed and compared to the level of galectin-3 in disease-free subjects. This is the first report to compare serum galectin-3 levels between prostate cancer patients and normal subjects, and find that plasma galectin-3 concentrations are significantly higher in patients with prostate cancer as compared to healthy subjects. Additionally, serum galectin-3 levels are at a peak in the most advanced stage of prostate cancer. Surprisingly, the galectin-3 levels in prostate cancer were increased over the levels in normal individuals, not decreased as previously thought. Additionally, galectin-3 levels demonstrate more consistent results in predicting the presence of prostate cancer than did PSA levels. PSA levels alone were not confirmatory of prostate cancer. The PSA levels for one prostate cancer patient (0.9) were lower than the levels in most of the healthy controls. The galectin-3 level of this patient, however, was higher than the levels in all healthy controls. Accordingly, the results indicated that galectin-3 can serve as a biomarker for the presence or absence of prostate cancer, either alone or in combination with PSA testing.

Before the present disclosure, the link between PSA cleavage of galectin-3 and its phosphorylation status was unknown. Surprisingly, the present disclosure demonstrates that the level of cleaved galectin-3 is higher in prostate cancer patients than normal controls. The present disclosure further demonstrates for the first time that the phosphorylation of Tyr-107 by c-Abl blocks cleavage of galectin-3 by PSA. Without being bound by theory, this phosphorylation then affects the extracellular functions of galectin-3, which in turn leads to increased angiogenesis, chemotaxis, and heterotypic aggregation. The present disclosure also demonstrates for the first time that PTEN dephosphorylates galectin-3 Tyr(P)-107. This dephosphorylation permits cleavage of galectin-3 and inhibits its function. Activation of c-Abl (phosphorylation) and loss of PTEN (dephosphorylation) occur during tumor formation and the progression of prostate cancer.

As a consequence, and without being bound by theory, the balance between phosphorylation and dephosphorylation is disrupted, galectin-3 is phosphorylated at Tyr-107 by cAbl and secreted, the secreted galectin-3 Tyr(P)-107 is resistant to cleavage by PSA, and is thus able to cross-link its cell surface ligands (multivalency) and trigger the initiation of cell surface molecule-associated signaling, which promotes the cancer behavior of the cells. In healthy individuals, by contrast, unphosphorylated galectin-3 is cleaved at Tyr-107 by PSA, resulting in removal of the N-terminal part of the protein, which thus blocks its ability to create the cross-linked lattices and promote the cancer behavior of the cells.

The present disclosure, in demonstrating the increase in overall galectin-3 levels and cleaved galectin-3 in prostate cancer patients, and the association of its cleavage and phosphorylation states with prostate cancer activity, provides further insights into biological aspects of prostate cancer tumor behavior. This includes not only the role of galectin-3, but also tPSA, the MMPs, c-Abl, and PTEN, for example.

Certain embodiments of the present disclosure, then, comprise methods wherein the level of galectin-3 in blood or other tissue is used as a marker for detecting the presence or absence of prostate cancer, or discriminating clinically low stage prostate cancer from more aggressive and higher stage tumors. Certain embodiments comprise a combination of detecting galectin-3 levels and PSA levels. Other embodiments of the present disclosure comprise methods of using galectin-3 blood levels, with or without PSA testing, to monitor the response to treatment for prostate cancer. In other embodiments galectin-3 is a therapeutic target for prostate cancer treatment.

Other embodiments of the present disclosure relate to methods wherein the ratio of cleaved to intact galectin-3 is a marker for the detection and/or monitoring of the presence or absence of prostate cancer. Other embodiments relate to methods wherein a determination of the ratio of phosphorylated to dephosphorylated galectin-3 is a marker for the detection and/or monitoring of the presence or absence of prostate cancer. The present disclosure further contemplates any combination of the foregoing in methods of detecting and/or monitoring the presence or absence of prostate cancer, alone or in combination with PSA testing. Note the methods of the present disclosure can further be used in patients with a low or high level of PSA in order to confirm the presence or absence of prostate cancer.

The foregoing methods of the present disclosure comprising determining galectin-3 levels, when combined with PSA levels, result in a reduction in the number of false positives and negative results that are seen for prostate cancer diagnosis when performing PSA testing alone.

Thus, disclosed herein are methods of diagnosing prostate cancer in a subject, methods of determining the stage of prostate cancer in a subject, methods of determining the effectiveness of a treatment regiment for prostate cancer in a subject, and methods of determining the progression of prostate cancer in a subject. In each of the methods, at least one biological sample is obtained from the subject and the levels of galectin-3, and optionally PSA, are determined in the sample. Methods for the determination of galectin-3 and/or PSA in a sample include, but are not limited to, immunoassays. In particular embodiments, the galectin-3 measured is intact galectin-3, cleaved galectin-3, phosphorylated galectin-3, unphosphorylated galectin-3, the galectin-3 1-107 fragment, or the galectin-3 108-250 fragment. In other embodiments, the PSA measured is free PSA (fPSA), PSA covalently complexed to α1-antichymotrypsin (PSA-ACT), or PSA covalently complexed to α2-macroglobulin (PSA-MG). As used herein, the term “cleaved galectin-3” refers to the 1-107 fragment of galectin-3, the 108-250 fragment of galectin-3, or the 1-107 fragment of galectin-3 and the 108-250 fragment of galectin-3.

In certain embodiments, the levels of galectin-3 (intact, cleaved, phosphorylated, and/or unphosphorylated), and optionally PSA, can be determined sequentially over time. In exemplary embodiments, galectin-3 or PSA levels can be determined 1 time, 2 times, 3 times, 4 times, 5 times, 6 times, 7 times, 8 times, 9 times, 10 times and every remaining integer up to 100 times or more. In a subject at risk of having prostate cancer, the galectin-3, and optionally PSA, levels can be determined weekly, monthly, every 2 months, every 3 months, every 4 months, every 5 months, every 6 months, every 7 months, every 8 months, every 9 months, every 10 months, or every 11 months, or yearly to determine if the subject has prostate cancer or to determine if a treatment has been effective or ineffective, or a prostate cancer is progressing or regressing (i.e., each measure can provide an intra-subject reference level). In a subject undergoing treatment for prostate cancer, the galectin-3, and optionally PSA, levels can be determined weekly, monthly, every 2 months, every 3 months, every 4 months, every 5 months, every 6 months, every 7 months, every 8 months, every 9 months, every 10 months, or every 11 months, or yearly to determine if the prostate cancer in the subject has progressed, has regressed, or has been successfully or unsuccessfully treated. In certain embodiments, a single determination of galectin-3, and optionally PSA, is used in the disclosed method. In certain embodiments, reference levels of galectin-3 and PSA are used.

In certain embodiments, the biological sample is blood, plasma, serum, urine, saliva, or semen.

Embodiments disclosed herein also include screening subjects who are at risk for prostate cancer. A subject at risk for prostate cancer can be a male over 50 years old, a male with a family history of prostate cancer, a male of certain race groups, or a male with a high fat diet. In other embodiments, the subject at risk of prostate cancer is a male older than 55 years, older than 60 years, older than 65 years, or older than 70 years.

A family history of prostate cancer refers to having a father, uncle, grandfather, or brother with prostate cancer. The risk is increased further if the family member is a brother, or more than one family member was affected by prostate cancer. In subjects with a family history of prostate cancer, the subject can be classified as at risk beginning at an age of age 35 years, 40 years, 45 years, 50 years, or 55 years.

A subject having increased risk of prostate cancer based on a racial classification includes (i) African-American men and (ii) Japanese-American men as compared to African and Japanese men living in their native countries. Rates for African and Japanese men increase sharply when they immigrate to the U.S. Subjects in these groups can be classified as at risk beginning at an age of age 35 years, 40 years, 45 years, 50 years, or 55 years.

In certain embodiments, a subject has prostate cancer, if (i) the level of galactin-3 in the biological sample is significantly elevated compared to the galactin-3 reference level; (ii) the level of phosphorylated galectin-3 is significantly elevated compared to the unphosphorylated galectin-3 level; (iii) the level of intact galectin-3 is significantly elevated compared to the cleaved galectin-3 level; (iv) the level of galactin-3 in the biological sample is significantly elevated compared to the galectin-3 reference level and the level of PSA in the biological sample is significantly elevated compared to the PSA reference level; (v) the level of intact galectin-3 is significantly elevated compared to the level of cleaved galectin-3; (vi) the level of intact galectin-3 is significantly elevated compared to the level of cleaved galectin-3 and the level of PSA is significantly elevated compared to the PSA reference level; (vii) the level of phosphorylated galectin-3 is significantly elevated compared to the level of unphosphorylated galectin-3; and/or (viii) the level of phosphorylated galectin-3 is significantly elevated compared to the level of unphosphorylated galectin-3 and the level of PSA is significantly elevated compared to the PSA reference level.

In certain embodiments, the prostate cancer therapy in a subject with prostate cancer is ineffective if (i) the level of galatin-3 in the biological sample is significantly elevated or unchanged compared to the galactin-3 reference level; (ii) the level of phosphorylated galectin-3 is significantly elevated or unchanged compared to the unphosphorylated galectin-3 level; (iii) the level of intact galectin-3 is significantly elevated or unchanged compared to the cleaved galectin-3 level; (iv) the level of galactin-3 in the biological sample is significantly elevated or unchanged compared to the galectin-3 reference level and the level of PSA in the biological sample is significantly elevated or unchanged compared to the PSA reference level; (v) the level of intact galectin-3 is significantly elevated or unchanged compared to the level of cleaved galectin-3; (vi) the level of intact galectin-3 is significantly elevated or unchanged compared to the level of cleaved galectin-3 and the level of PSA is significantly elevated or unchanged compared to the PSA reference level; (vii) the level of phosphorylated galectin-3 is significantly elevated or unchanged compared to the level of unphosphorylated galectin-3; and/or (viii) the level of phosphorylated galectin-3 is significantly elevated or unchanged compared to the level of unphosphorylated galectin-3 and the level of PSA is significantly elevated or unchanged compared to the PSA reference level. In certain embodiments, the results of the method are indicative of a need to change the therapy regimen in the subject, or are an indication of progression of the disease. Thus, the disclosed methods can be used to monitor the efficacy of a particular treatment regimen in a subject and recommend a change in treatment regimen prior to onset of clinical symptoms of progression or relapse.

In certain embodiments, the method is useful for determining if prostate cancer has progressed in the subject, or if a subject previously treated for prostate cancer has relapsed, wherein the prostate cancer has progressed if (i) the level of galatin-3 in the biological sample is significantly elevated compared to the galactin-3 reference level; (ii) the level of phosphorylated galectin-3 is significantly elevated compared to the unphosphorylated galectin-3 level; (iii) the level of intact galectin-3 is significantly elevated compared to the cleaved galectin-3 level; (iv) the level of galactin-3 in the biological sample is significantly elevated compared to the galectin-3 reference level and the level of PSA in the biological sample is significantly elevated compared to the PSA reference level; (v) the level of intact galectin-3 is significantly elevated compared to the level of cleaved galectin-3; (vi) the level of intact galectin-3 is significantly elevated compared to the level of cleaved galectin-3 and the level of PSA is significantly elevated compared to the PSA reference level; (vii) the level of phosphorylated galectin-3 is significantly elevated compared to the level of unphosphorylated galectin-3; and/or (viii) the level of phosphorylated galectin-3 is significantly elevated compared to the level of unphosphorylated galectin-3 and the level of PSA is significantly elevated compared to the PSA reference level.

Reductions in the described measures can also indicate spontaneous remission of the disease and/or the effectiveness of a treatment regimen.

A used herein, the term “significantly elevated” with regard to changes in galectin-3 or PSA levels can refer to an increase of more than 20%, more than 30%, more than 40%, more than 50%, more than 60%, more than 70%, more than 80%, more than 90%, more than 100%, more than 150%, or more than 200% compared to a reference level. The galactin-3 is intact galectin-3, cleaved galectin-3, phosphorylated galectin-3, unphosphorylated galectin-3, galectin-3 fragment 1-107, or galectin-3 fragment 108-250. As used herein, “significantly elevated” measures can be evaluated independently against a reference level without consideration of earlier comparisons in the same subject.

As used herein, “unchanged” measures are evaluated in relation to a previous comparison in the same subject and denote a failure to achieve a statistically significant change in a score towards or away from a reference level in the particular subject.

In some embodiments of the present teachings, results of the methods disclosed herein can be used to direct a subject's treatment. For example, the results of the methods can be used to assess subjects for primary diagnosis, and/or for disease management. For the primary diagnosis, the results can be used for prediction and risk stratification for progression of the condition or disease sequelae, for the diagnosis of prostate cancer, for the rate of change of prostate cancer, and/or for indications for future diagnosis and therapeutic regimens. For disease management, the results can be used for prognosis and risk stratification. The results of the methods can be used for clinical decision support, such as determining whether to defer intervention or treatment, to recommend preventive check-ups for at-risk patients, to recommend increased visit frequency, to recommend increased testing, and/or to recommend intervention. The results of the methods can also be useful for therapeutic selection, determining response to treatment, adjustment and dosing of treatment, monitoring ongoing therapeutic efficiency, and indication for change in therapeutic regimens.

In various embodiments, the methods and kits described herein can be used to detect the presence or absence of non-aggressive prostate cancer, aggressive prostate cancer, non-metastatic prostate cancer, and/or metastatic prostate cancer. In various embodiments, the methods and kits described herein can be used in relation to the treatment of non-aggressive prostate cancer, aggressive prostate cancer, non-metastatic prostate cancer, and/or metastatic prostate cancer.

The present disclosure further provides for a kit comprising one or more detection assays for practicing any of the methods disclosed herein. The kits may include a notice in the form prescribed by a governmental agency regulating the manufacture, use or sale of pharmaceuticals, biological products, lab developed tests, etc., which notice reflects approval by the agency of the manufacture, use or sale for human administration and/or testing. The detection assays of the kits may utilize any necessary or appropriate polypeptides, conjugates, antibodies, polynucleotides, expression vectors, cells, methods, compositions, systems, and/or apparatuses useful for the detection of galectin-3, intact galectin-3, cleaved galectin-3, phosphorylated galectin-3, unphosphorylated galectin-3, and PSA. The kits may also include instructions for practicing any method described herein.

In certain embodiments of the kit, the kit includes one of more of detection assays for detecting intact galectin-3, cleaved galectin-3, phosphorylated galectin-3, unphosphorylated galectin-3, galectin-3 1-107, galectin-3 108-250, and PSA. In other embodiments, the kit includes three or more detection assays, four or more detection assays, five or more detection assays, six or more detection assays, seven or more detection assays, or eight or more detection assays. The kit further includes all, most, or some of the reagents necessary for performing the assays. Persons of ordinary skill in the art will understand that certain reagents would be available at the testing locations such as water, saline, etc. and that these reagents might not be included in the kit.

The kit further comprises reference levels, or instructions on determining reference levels. Optionally reference levels are determined using the detection kits.

Some embodiments disclosed herein relate to methods of determining the presence or absence of prostate cancer in a subject, comprising determining a galectin-3 level in a biological sample obtained from the subject, obtaining a first dataset comprising the galectin-3 level, inputting the first dataset into an interpretation function that transforms the first dataset into a score, and determining whether the subject has prostate cancer based on the score. In certain embodiments, an increase in galectin-3 level is associated with the presence of prostate cancer in the subject.

One embodiment includes a method of determining the presence or absence of prostate cancer in a subject comprising: determining a galectin-3 level in a biological sample obtained from the subject; comparing the measured galectin-3 level with a reference level; and determining prostate cancer is present in the subject if the level of galectin-3 in the biological sample is significantly elevated compared to the reference level.

Another embodiment includes a method of determining the presence or absence of prostate cancer in a subject comprising: determining a galectin-3 level in a biological sample obtained from the subject; determining a PSA level in a biological sample obtained from the subject; comparing the measured galectin-3 level with a galectin-3 reference level; comparing the measured PSA level with a PSA reference level; and determining prostate cancer is present in the subject if (i) the level of galectin-3 in the biological sample is significantly elevated compared to the galectin-3 reference level or (ii) the level of galectin-3 in the biological sample is significantly elevated compared to the galectin-3 reference level and the level of PSA in the biological sample is significantly elevated compared to the PSA reference level.

Another embodiment includes a method of determining the presence or absence of prostate cancer in a subject comprising: determining a galectin-3 level in a biological sample obtained from the subject; determining a PSA level in a biological sample obtained from the subject; creating a weighted score based on the determined levels with the level of galectin-3 weighted more heavily than the level of PSA; comparing the weighted score with a reference weighted score; and determining prostate cancer is present in the subject if the weighted score is significantly elevated from the reference weighted score.

In one embodiment, biomarker results are weighted based upon known diagnostic criteria and/or patient history, lifestyle, symptoms, and the like. The resulting aggregate weighted score can be used for clinical assessment.

In another embodiment, weighting scores involves converting the measurement of one biomarker that is identified and quantified in a test sample into one of many potential scores. A ROC curve can be used to standardize the scoring between different markers by enabling the use of a weighted score based on the inverse of a false positive % or false negative % defined from the ROC curve. The weighted score can be calculated by multiplying the AUC by a factor for a marker and then dividing by the false positive % or false negative % based on a ROC curve. The weighted score can be calculated using the formula:

Weighted Score=(AUCx×factor)/(1−% specificityₓ)

wherein x is the marker; the, “factor,” is a real number (such as 0, 1, 2, 3, 4, 5, 6, 7, 8, 9, 10, 11, 12, 13, 14, 15, 16, 17, 18, 19, 20, 21, 22, 23, 24, 25 and so on) throughout a panel; and the specificity is a chosen value that does not exceed 95%. Multiplication of a factor for the panel allows the user to scale the weighted score.

Another embodiment includes a method of determining the effectiveness of a treatment regimen for prostate cancer comprising: determining a galectin-3 level in a biological sample obtained from the subject; comparing the measured galectin-3 level with a reference level; and determining the treatment regimen is ineffective if the level of galectin-3 in the biological sample is significantly elevated or unchanged compared to the reference level.

Another embodiment includes a method of determining the effectiveness of a treatment regimen for prostate cancer comprising: determining a galectin-3 level in a biological sample obtained from the subject; determining a PSA level in a biological sample obtained from the subject; comparing the measured galectin-3 level with a galectin-3 reference level; comparing the measured PSA level with a PSA reference level; and determining the treatment regimen is ineffective if (i) the level of galectin-3 in the biological sample is significantly elevated or unchanged compared to the galectin-3 reference level, or (ii) the level of galectin-3 in the biological sample is significantly elevated or unchanged compared to the galectin-3 reference level and the level of PSA in the biological sample is significantly elevated or unchanged compared to the PSA reference level.

Another embodiment includes a method of determining the effectiveness of a treatment regimen for prostate cancer comprising: determining a galectin-3 level in a biological sample obtained from the subject; determining a PSA level in a biological sample obtained from the subject; creating a weighted score based on the determined levels with the level of galectin-3 weighted more heavily than the level of PSA; comparing the weighted score with a reference weighted score; and determining the treatment regimen is ineffective if the weighted score is significantly elevated from, the reference weighted score and/or not significantly changed from a previous comparison to the reference weighted score.

Another embodiment includes a method of determining the presence or absence of prostate cancer in a subject comprising: determining levels of cleaved galectin-3 and intact galectin-3 in a biological sample obtained from the subject; comparing the amounts of cleaved galectin-3 and intact galectin-3 in the biological sample; and determining prostate cancer is present in the subject if the level of intact galectin-3 is significantly elevated compared to the level of cleaved galectin-3.

Another embodiment includes a method of determining the presence or absence of prostate cancer in a subject comprising: determining levels of cleaved galectin-3 and intact galectin-3 in a biological sample obtained from the subject; determining a PSA level in a biological sample obtained from the subject; comparing the levels of cleaved galectin-3 versus intact galectin-3; comparing the measured PSA level with a PSA reference level; and determining prostate cancer is present in the subject if (i) the level of intact galectin-3 in the biological sample is significantly elevated compared to the level of cleaved galectin-3 or (ii) the level of intact galectin-3 in the biological sample is significantly elevated compared to the level of cleaved galectin-3 and the level of PSA in the biological sample is significantly elevated compared to the PSA reference level.

Another embodiment includes a method of determining the presence or absence of prostate cancer in a subject comprising: determining levels of cleaved galectin-3 and intact galectin-3 in a biological sample obtained from the subject; determining a PSA level in a biological sample obtained from the subject; creating a weighted score based on the determined levels of (i) intact galectin-3 compared to cleaved galectin-3 and (ii) PSA, with the intact galectin-3/cleaved galectin-3 ratio weighted more heavily than the level of PSA; comparing the weighted score with a reference weighted score; and determining prostate cancer is present in the subject if the weighted score is significantly elevated from the reference weighted score.

Another embodiment includes a method of determining the effectiveness of a treatment regimen for prostate cancer comprising: determining levels of cleaved galectin-3 and intact galectin-3 in a biological sample obtained from the subject; comparing the amounts of cleaved galectin-3 and intact galectin-3 in the biological sample; and determining the treatment regimen is ineffective if the level of intact galectin-3 in the biological sample is significantly elevated compared to the level of cleaved galectin-3.

Another embodiment includes a method of determining the effectiveness of a treatment regimen for prostate cancer comprising: determining levels of cleaved galectin-3 and intact galectin-3 in a biological sample obtained from the subject; determining a PSA level in a biological sample obtained from the subject; comparing the levels of cleaved galectin-3 versus intact galectin-3; comparing the measured PSA level with a PSA reference level; and determining the treatment regimen is ineffective if (i) the level of intact galectin-3 in the biological sample is significantly elevated compared to the level of cleaved galectin-3 or (ii) the level of intact galectin-3 in the biological sample is significantly elevated compared to the level of cleaved galectin-3 and the level of PSA in the biological sample is significantly elevated from, the reference weighted score and/or not significantly changed from a previous comparison to the reference weighted score.

Another embodiment includes a method of determining the effectiveness of a treatment regimen for prostate cancer comprising: determining levels of cleaved galectin-3 and intact galectin-3 in a biological sample obtained from the subject; determining a PSA level in a biological sample obtained from the subject; creating a weighted score based on the determined levels of (i) cleaved galectin-3 versus intact galectin-3 levels and (ii) PSA, with the cleaved galectin-3/intact galectin-3 ratio weighted more heavily than the level of PSA; comparing the weighted score with a reference weighted score; and determining the treatment regimen is ineffective if the weighted score is significantly different from or unchanged from the reference weighted score.

In another embodiment, the cleaved galectin-3 comprises the 1-107 fragment of galectin-3, the 108-250 fragment of galectin-3, or the 1-107 fragment of galectin-3 and the 108-250 fragment of galectin-3.

Another embodiment includes a method of determining the presence or absence of prostate cancer in a subject comprising: determining levels of phosphorylated galectin-3 and unphosphorylated galectin-3 in a biological sample obtained from the subject; comparing the amounts of phosphorylated galectin-3 and unphosphorylated galectin-3 in the biological sample; and diagnosing the subject with prostate cancer if the level of phosphorylated galectin-3 is significantly elevated compared to the level of unphosphorylated galectin-3.

Another embodiment includes a method of determining the presence or absence of prostate cancer in a subject comprising: determining levels of phosphorylated galectin-3 and unphosphorylated galectin-3 in a biological sample obtained from the subject; determining a PSA level in a biological sample obtained from the subject; comparing the levels of phosphorylated galectin-3 versus unphosphorylated galectin-3; comparing the measured PSA level with a PSA reference level; and diagnosing the subject with prostate cancer if (i) the level of phosphorylated galectin-3 in the biological sample is significantly elevated compared to the level of unphosphorylated galectin-3 or (ii) the level of phosphorylated galectin-3 in the biological sample is significantly elevated compared to the level of unphosphorylated galectin-3 and the level of PSA in the biological sample is significantly elevated compared to the PSA reference level.

Another embodiment includes a method of determining the presence or absence of prostate cancer in a subject comprising: determining levels of phosphorylated galectin-3 and unphosphorylated galectin-3 in a biological sample obtained from the subject; determining a PSA level in a biological sample obtained from the subject; creating a weighted score based on the determined levels of (i) phosphorylated galectin-3 versus unphosphorylated galectin-3 levels and (ii) PSA, with the phosphorylated galectin-3/unphosphorylated galectin-3 ratio weighted more heavily than the level of PSA; comparing the weighted score with a reference weighted score; and determining prostate cancer is present in the subject if the weighted score is significantly elevated from the reference weighted score.

Another embodiment includes a method of determining the effectiveness of a treatment regimen for prostate cancer comprising: determining levels of phosphorylated galectin-3 and unphosphorylated galectin-3 in a biological sample obtained from the subject; comparing the amounts of phosphorylated galectin-3 and unphosphorylated galectin-3 in the biological sample; and determining the treatment regimen is ineffective if the level of phosphorylated galectin-3 in the biological sample is significantly elevated from the level of unphosphorylated galectin-3 and/or not significantly changed from a previous comparison to the unphosphorylated galectin-3.

Another embodiment includes a method of determining the effectiveness of a treatment regimen for prostate cancer comprising: determining levels of phosphorylated galectin-3 and unphosphorylated galectin-3 in a biological sample obtained from the subject; determining a PSA level in a biological sample obtained from the subject; comparing the levels of phosphorylated galectin-3 and unphosphorylated galectin-3; comparing the measured PSA level with a PSA reference level; and determining the treatment regimen is ineffective if (i) the level of phosphorylated galectin-3 in the biological sample is significantly elevated compared to the level of unphosphorylated galectin-3, or (ii) the level of phosphorylated galectin-3 in the biological sample is significantly elevated compared to the level of unphosphorylated galectin-3 and the level of PSA in the biological sample is significantly elevated compared to the PSA reference level or unchanged from a previous comparison to the PSA reference level.

Another embodiment includes a method of determining the effectiveness of a treatment regimen for prostate cancer comprising: determining levels of phosphorylated galectin-3 and unphosphorylated galectin-3 in a biological sample obtained from the subject; determining a PSA level in a biological sample obtained from the subject; creating a weighted score based on the determined levels of (i) phosphorylated galectin-3 versus unphosphorylated galectin-3 levels, and (ii) PSA with the phosphorylated galectin-3/unphosphorylated galectin-3 ratio weighted more heavily than the level of PSA; comparing the weighted score with a reference weighted score; and determining the treatment regimen is ineffective if the weighted score is significantly elevated from, the reference weighted score and/or not significantly changed from a previous comparison to the reference weighted score.

In another embodiment, the galectin-3 is phosphorylated at position Tyr-107 of galectin-3.

In another embodiment, the reference level, galectin-3 reference level, PSA reference level and/or reference weighted score is derived from (i) an individual who does not have prostate cancer, (ii) a group of individuals who do not have prostate cancer; (iii) the subject before diagnosis with prostate cancer or (iv) the subject at the beginning of a treatment regimen for prostate cancer.

In another embodiment, the level of galectin-3, the cleaved galectin-3/intact galectin-3 ratio or the phosphorylated galectin-3/unphosphorylated galectin-3 ratio is weighted at least twice as heavily as the level of PSA in creating the weighted score; at least three times as heavily as the level of PSA in creating the weighted score; at least four times as heavily as the level of PSA in creating the weighted score; or at least five times as heavily as the level of PSA in creating the weighted score.

The present disclosure also includes kits that can be used to carry out any of the embodiments disclosed herein.

In one embodiment, the kit comprises at least one galectin-3 detection assay.

In another embodiment, the kit comprises at least one galectin-3 detection assay and at least one PSA detection assay.

In another embodiment, the kit comprises at least one intact galectin-3 detection assay and at least one cleaved galectin-3 assay.

In another embodiment, the kit comprises at least one intact galectin-3 detection assay, at least one cleaved galectin-3 assay and at least one PSA detection assay.

In another embodiment, the kit comprises at least one phosphorylated galectin-3 detection assay and at least one unphosphorylated galectin-3 assay.

In another embodiment, the kit comprises at least one phosphorylated galectin-3 detection assay, at least one unphosphorylated galectin-3 assay and at least one PSA detection assay.

In another embodiment of the kits, the detection assays can provide quantitative measurements of the detected galectin-3, detected cleaved galectin-3, detected intact galectin-3, detected phosphorylated galectin-3, detected unphosphorylated galectin-3 and/or detected PSA.

### EXAMPLES

### Example 1

The present Example demonstrates that phosphorylation by c-Abl at the Tyr-107 residue of galectin-3 blocks its cleavage by PSA, and affects the extracellular functions of galectin-3, leading to increased angiogenesis, chemotaxis, and heterotypic aggregation. This Example also shows that dephosphorylation of galectin-3 Tyr(p)-107 by phosphatase and tensin homologue (PTEN; which is deleted on chromosome 10 and frequently down-regulated in progressive prostate cancer, associated with a gain in galectin-3 function and oncogenic signaling), allows the cleavage by PSA and inhibits galectin-3 function.

Cell Lines and Antibodies. The human prostate cancer cell line LNCaP C4-2B (LNCaP) was purchased from Urocor (Oklahoma City, Okla.) and maintained in RPMI 1640 medium (Invitrogen) supplemented with 10% fetal bovine serum (FBS; Atlanta Biologicals). Bovine adrenal microvascular endothelial cells (BAMEC) were a gift from Dr. D. Banerjee (University of Puerto Rico, San Juan, Puerto Rico) and cultured in medium consisting of minimal essential medium with Earle's salts and L-glutamine (Invitrogen) supplemented with 10% FBS (HyClone) and antibiotics (Mediatech). Human prostate cancer PC3M cells were a gift from Dr. Isaiah Fidler (University of Texas MD Anderson Cancer Center, Houston, Tex.) and cultured in Dulbecco's modified Eagle's medium (DMEM) supplemented with 10% FBS (Atlanta Biologicals). All cells were maintained in a humidified chamber with 95% air and 5% CO₂ at 37° C. The cells were grown to near confluence and detached from the monolayer with 0.25% trypsin and 2 mM EDTA for 2 min at 37° C. The use of cell lines was approved by the Human Investigation Committee, Wayne State University, Detroit, Mich.

Customized polyclonal rabbit anti-galectin-3 antibody against the recombinant whole molecule was created by Zymed Laboratories Inc. Monoclonal rat anti-galectin-3 M3/38 antibody was isolated from the supernatant of hybridoma TIB-166 (American Type Culture Collection). Phospho-tyrosine blot was performed with anti-Tyr(P) antibody coupled with IRDye 800 (Rockland Immunochemicals). Mouse anti-β-actin antibody was purchased from Sigma-Aldrich.

Plasmid Constructs and Purification of Recombinant Proteins. Galectin-3 was PCR-amplified from a pcDNA-Gal-3 wild type vector and subcloned into pVitB (modified pcDNA6) as a BamHI-EcoRI fragment. Full-length galectin-3 and 1-107 and 108-250 human galectin-3 were subcloned into the pET30as (modified pET30a) vector as a BamHI-XhoI fragment and overexpressed in Escherichia coli at 20° C. The expression construct introduced a His-tag to the protein. The soluble protein was purified by nickel-agarose affinity chromatography. The protein was concentrated in a buffer containing 20mMTris (pH 7.9), 25% (v/v) glycerol, 10 mM DTT, 2 mM EDTA and stored at −80° C. The His tags were not removed from the protein.

c-Abl Kinase Assay. The c-Abl kinase assay was performed using the HTScan Abl1 kinase assay kit (Cell Signaling Technology) according to the manufacturer's instructions.

Three-dimensional Growth and Tube Formation Assay. To analyze in vitro angiogenesis and interactions between epithelial and endothelial cell formation of tubular networks by co-cultured BAMEC and LNCaP cells, a three-dimensional growth and tube formation assay was performed. Briefly, MATRIGEL® (10 μl) was added to each chamber of the slide (μ-Slide, ibidi, Martinsried, Germany) and gelled by a 30-min incubation at room temperature, after which 15,000 BAMEC and 5000 LNCaP cells were plated onto the gel in 50 μl of Eagle's minimal essential complete medium. In some chambers, full-length galectin-3 or its fragments were added. After 36 hr, three-dimensional structures were observed under phase contrast microscope and photographed. Images were acquired with an Olympus IX71 microscope supporting a Hamamatsu ORCA-ER video camera. Tube formation image analysis was done using the web-based Image Analysis Win-Tube module of Wimasis online software.

Western Blot Analysis. Cells were grown up to 80% confluence, and whole-cell lysates were prepared in lysis buffer (20 mM Tris-HCl (pH 7.4), 0.1% SDS, 1.0% Triton X-100, 0.25% sodium deoxycholate, 1 mM EGTA, 1 mM EDTA, 5 mM sodium fluoride, 1 mM sodium orthovanadate, 1 mM phenylmethylsulfonyl fluoride, 1 μg/ml leupeptin, 1 μg/ml pepstatin and 1 μg/ml aprotinin (Sigma). An equal amount of protein was loaded on the gel, resolved by 10% SDS-PAGE, and electroblotted onto polyvinylidene difluoride membrane (IMMOBILON® FL, Millipore). Membranes were quenched in a solution of TBS containing 0.1% casein and 0.1% Tween 20 for 60 min or in 2% gelatin (teleostean gelatin) in the case of phospho-blots (Sigma) in TBST (TBS containing 0.1% Tween 20) on a rotary shaker. Blots were incubated with the appropriate primary antibodies, washed, and then incubated with the appropriate secondary antibodies conjugated with IRDye 800 (Rockland Immunochemicals) or Alexa Fluor 680 (Invitrogen) for 30 min at room temperature. After incubation with both the primary and the secondary antibodies, membranes were washed four times with TBST at 5-min intervals. Immunoblots were visualized, and the density of each band was quantitated using the Odyssey infrared imaging system and Odyssey application software (LI-COR Biosciences).

Chemotaxis. This assay was performed using a Boyden chamber (Neuroprobe). In the lower chamber, the chemo-attractant MATRIGEL® (BD Biosciences) alone or mixed with various concentrations of recombinant galectin-3, galectin-3 1-107, or galectin-3 108-250 was added. LNCaP (5×10⁴) cells suspended in basic DMEM were loaded in the upper chamber. The two chambers were separated by a polycarbonate filter of 5-micron pore size and incubated in a 37° C. tissue culture incubator for 5 hr, after which the filter was removed, the cells on top of the filter were wiped off, and the migrated cells were fixed, stained using Protocol Hema 3 stain set (Fisher Scientific), and counted under microscope. Each assay was carried out in triplicate.

Wound-healing Assay. A wound-healing assay, the scratch assay, was performed according to the protocol published in Liang, et al. (2007), Nat. Protoc. 2, 329-333. Briefly, in this scratch assay 60-mm dishes were coated with ECM and incubated overnight, unbound ECM was removed and the dishes were blocked with bovine serum albumin. Subconfluent growing cells were resuspended in a tissue culture dish by washing cells with PBS, adding versene containing trypsin, and mixing cells with medium containing serum. Cells were then plated onto the prepared 60-mm dish to create a confluent monolayer, and incubated for 6 hr at 37° C.

The cell lines LNCaP (null galectin-3), DU145 (stable clone with reduced galectin-3 expression), and PC3M were used to examine the effect of full-length galectin-3, galectin-3 1-107, and galectin-3 108-250 on prostate cancer cell motility using the scratch assay. The assay was performed with LNCaP, Du145, and PC3M cultured cell lines stimulated by full-length galectin-3, and fragments galectin-3 1-107 and 108-250. A uniform wound was made in each plate using a 1-μl pipette tip. The wound area was observed immediately, then at 24 hr after creation the cells were counted. Cells were grown under identical conditions.

Statistical Analysis. Data experiments are expressed as mean +/− S.D. of three independent experiments. Comparisons between the groups were determined by using the one-way analysis of variance test using an on-line calculator, where p<0.05 was considered statistically significant.

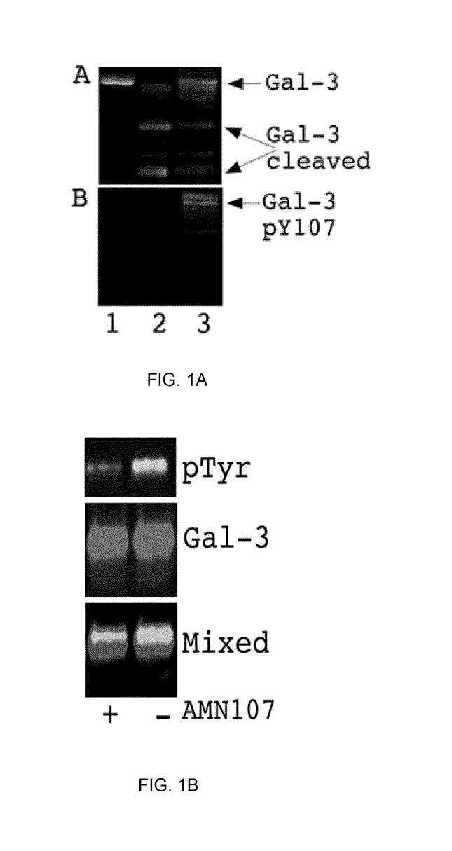

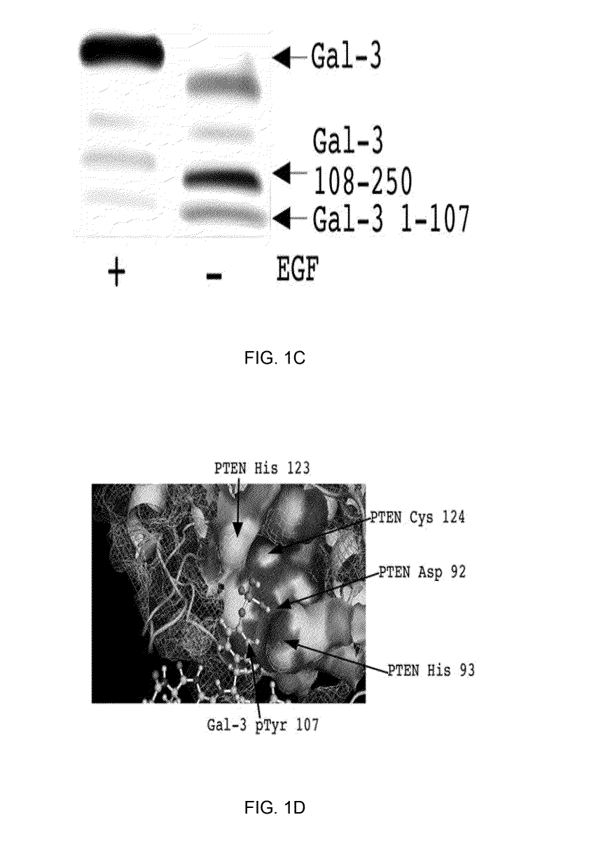

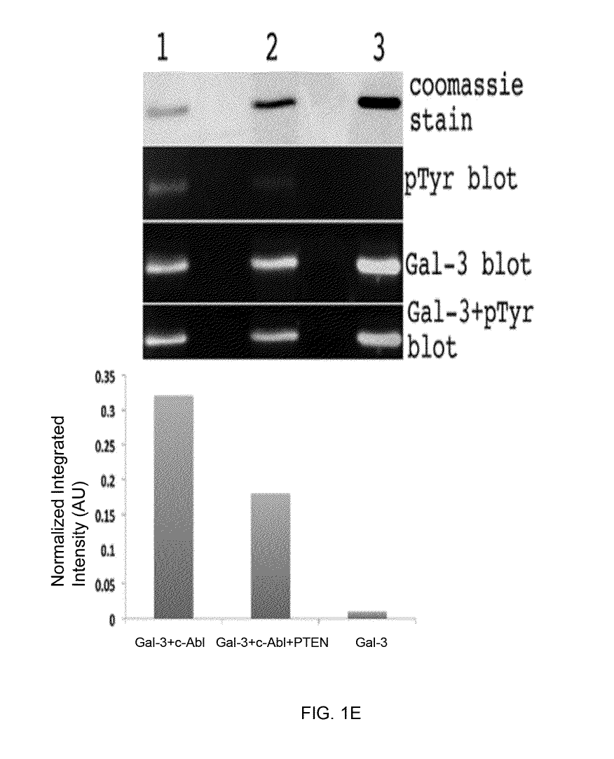

Phosphorylation of galectin-3 on Tyr-107 regulates its cleavage by PSA. Because Tyr-107 is the major phosphorylation site of galectin-3, in this Example the ability of active PSA to cleave galectin-3 phosphorylated on Tyr-107 was evaluated. As described above, wild type galectin-3 was purified and used as a substrate for active c-Abl kinase in an in vitro assay. Active PSA was added to untreated (unphosphorylated) and treated (phosphorylated) galectin-3, and after incubation at room temperature for 2 hours, the presence of cleaved protein and the phosphorylation status of galectin-3 were evaluated by Western blot analysis. An equal amount of recombinant galectin-3 wild type was loaded on the gel. The reaction was stopped by adding sample buffer, resolved on a 10% SDS-PAGE gel, and immunoblotted using anti-galectin-3 antibody or anti-Tyr(P) (pTyr) antibody. FIG. 1A demonstrated that phosphorylation of Tyr-107 blocks the cleavage of galectin-3 by PSA.

Tyrosine phosphorylation of galectin-3 in vivo is blocked by c-Abl inhibitor. See FIG. 1B. PC3M cells were treated with 150 ng/ml epidermal growth factor (EGF) and 50 ng/ml platelet derived growth factor (PDGF) for 8 hr. One plate was also treated with 1 μM AMN107 (c-Abl inhibitor). Conditioned media were collected, and galectin-3 was immunoprecipitated with TIB166 antibody. Samples were resolved using 10% SDS-PAGE gel and immunoblotted with anti-phosphotyrosine antibody (see FIG. 1B, pTyr) and anti-galectin-3 (HL31) antibody (Gal-3). The mixed lanes represent overlapping Tyr(P) and galectin-3 blots loaded and run on an SDS-PAGE gel.

The results demonstrated that after treatment of cells with EGF and PDGF, tyrosine phosphorylation can be detected on secreted galectin-3. The c-Abl inhibitor AMN107 significantly blocked galectin-3 tyrosine phosphorylation, indicating that this is primarily a c-Abl-dependent event.

Galectin-3 phosphorylated on Tyr-107 in vivo blocks the cleavage of secreted galectin-3 by PSA. See FIG. 1C. As none of the prostate cancer cell lines tested in this Example secreted phosphorylated galectin-3 or endogenous PSA, PC3M cells (which secreted galectin-3 Tyr(P)-107 after EGF treatment) were co-cultured with LNCaP cells (which secreted endogenous PSA), and the conditioned medium was collected after EGF treatment, as follows: LNCaP and PC3M cells were co-cultured for 24 hours. One plate was treated with 150 ng/ml EGF for 8 hours and conditioned medium was collected and concentrated using Millipore filters with a 3-kDa cutoff. Fifty micrograms of total protein was loaded and run on a gradient (4-20%) SDS-PAGE gel. After transfer, the membrane was blotted with polyclonal HL31 antibody, which can recognize multiple epitopes on galectin-3, for 2 hours at room temperature.

The results of these in vitro experiments indicated that phosphorylation blocks cleavage of galectin-3 in conditioned medium.

In FIG. 1D, a computer-generated visualization of the docking of Gal-3 phosphorylated on Tyr-107 with PTEN is presented. Autodock4 (Scripps Research Institute) was used to “dock” the phosphorylated version of galectin-3 in PTEN. The PTEN His-123, Cys-124, Asp-92, and His-93 shown in FIG. 1D represent amino acids from the catalytic active site of PTEN, and form the PTEN HCXXGXXR motif. The Cys-124 and Arg-130 residues are essential for catalysis, and the His-123 residue is important for the conformation of the P loop.

FIG. 1F depicts the dephosphorylation of galectin-3 Tyr(P)-107 with PTEN. In lane 1 is wild type recombinant galectin-3 phosphorylated with c-Abl. In lane 2 is phosphorylated galectin-3 that was treated with PTEN for 2 hr at 30° C. In lane 3 is recombinant, untreated galectin-3. The top panel represents 15% of the sample mixture run on 10% SDS-PAGE and visualized with Coomassie Blue stain. The rest of the samples were resolved using a 10% SDS-PAGE gel and immunoblotted with anti-phosphotyrosine antibody (labeled “pTyr blot”), anti-galectin-3 (HL31) antibody (labeled “Gal-3 blot”) and overlapping Tyr(P) and galectin-3 blots (labeled “Gal-3+pTyr blot”). The normalized integrated intensity (FIG. 1E) was calculated as band integrated intensity, where AU is arbitrary units.

Before the present disclosure, no one had demonstrated that a tyrosine phosphatase can dephosphorylate galectin-3 tyrosine residues. The present disclosure predicted from in silico docking (see FIG. 1D), that PTEN is one of the tyrosine phosphatases responsible for galectin-3 dephosphorylation. This was then proven by phosphorylating galectin-3 in vitro with active c-Abl and then treating it with PTEN. See FIG. 1E. The results suggest that PTEN is responsible for dephosphorylation of galectin-3 Tyr(P)-107 in vivo.

Functional significance of galectin-3 cleavage by PSA. Cleavage of galectin-3 with PSA creates a mixture of two galectin-3 fragments: residues 1-107 containing the NH₂-terminal domain with a repeated collagen-like sequence, and residues 108-250 which contains the functional carbohydrate recognition domain of galectin-3. To understand the physiological relevance of galectin-3 cleavage by PSA, peptides were constructed of the amino acid sequences resulting from cleavage at Tyr-107, and fused with His tags for purification using nickel-agarose affinity chromatography. Functional assays with which galectin-3 is involved were then performed.

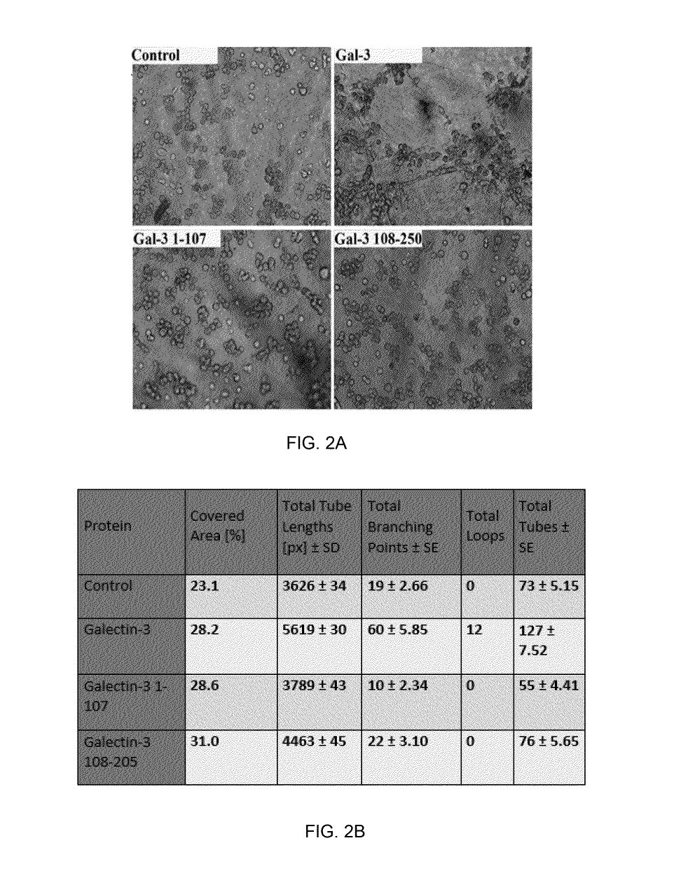

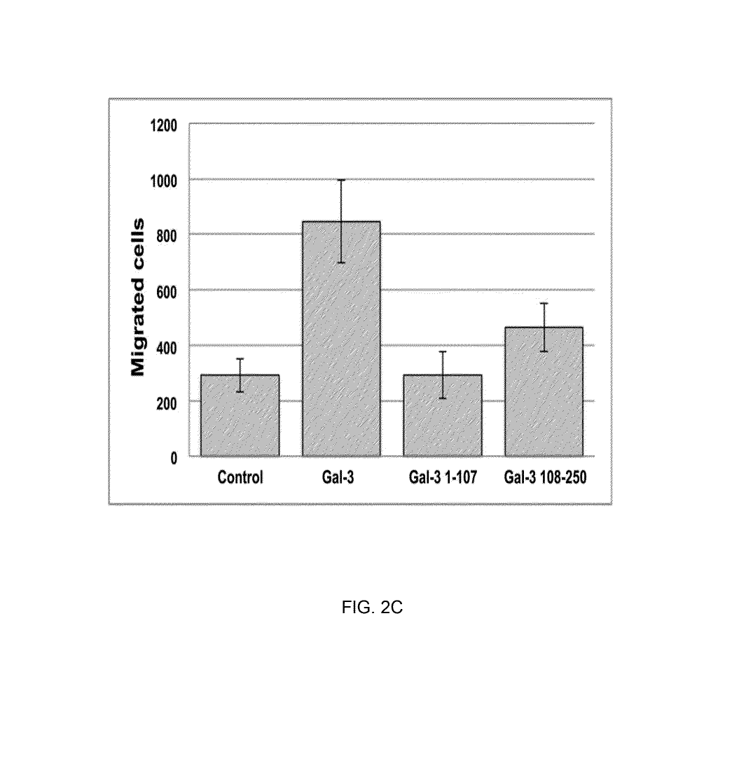

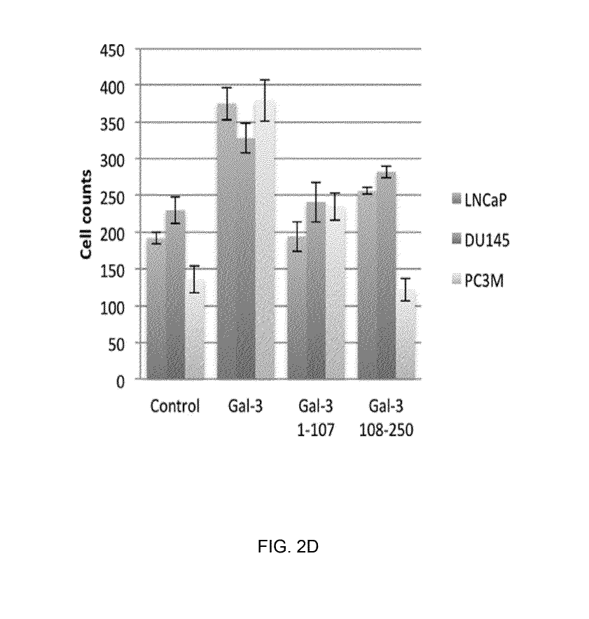

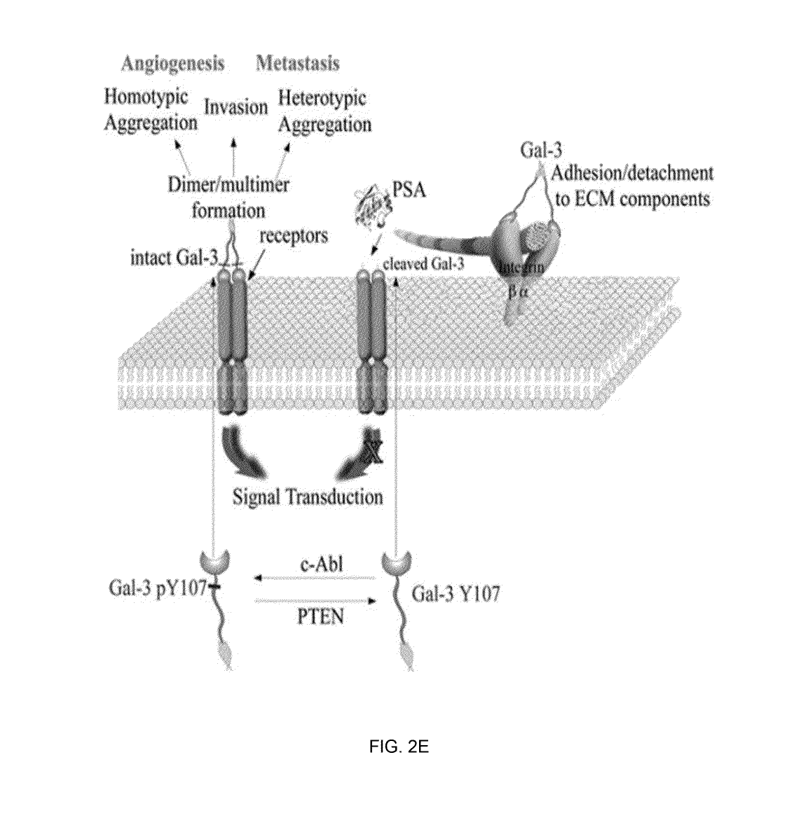

Three-dimensional heterotypic co-cultures of epithelial (LNCaP) and endothelial (BAMEC) cells were performed on MATRIGEL® to study tube formation in the presence of 10 μg/ml galectin-3 and its fragments. See FIG. 2A. Full-length galectin-3 added to the medium showed 127 units of tubes and 12 loops, respectively, as compared with 73,55, and 76 units of tubes and 0 loops by the control, the 1-107 fragment, and the 108-250 fragment, respectively. FIG. 2B presents a quantitative evaluation of the tube formation assay. Although galectin-3 induced branching morphogenesis of endothelial cells, galectin-3 1-107 and 108-250 failed to do so.

The effects of galectin-3 1-107 and 108-250 fragments, as well as full-length galectin-3 on migration of LNCaP cells in a chemotaxis assay were also tested. A Boyden chamber-based cell migration assay, in which the cells must penetrate a porous polycarbonate filter in moving toward a chemoattractant, demonstrated that migration of LNCaP cells toward MATRIGEL® with added full-length galectin-3 (10 μg/ml) was significantly higher as compared with control (wells without recombinant protein) or either of the two fragments of galectin-3. See FIG. 2C.

The effects of full-length galectin-3, galectin-3 1-107, and galectin-3 108-250 on prostate cancer cell motility was also examined using the scratch assay, as described above. As shown in FIG. 2D, only the addition of full-length galectin-3 induced endothelial cell chemotaxis, facilitating their motility during the initial phase of tube formation, whereas the fragments galectin-3 1-107 and 108-250 did not show a significant increase in motility as compared with the control. These results demonstrate that only full-length galectin-3 promotes prostate cancer cell motility.

In summary, galectin-3's cleavage state is important for various biological phenomena, including cell growth and morphogenesis and motility, and metastasis. Only full-length galectin-3 functions as the factor promoting chemotaxis, morphogenesis, and cell motility.

### Example 2

Subjects. Men were recruited from the Karmanos Cancer Institute during their visit to the Institute after signing a consent form. Eligibility criteria were that subjects be at least 18 years of age. A total of 16 subjects were enrolled. Of the 16 subjects, eight were prostate cancer patients and the other eight were disease-free, and were randomly selected to match the age distribution of the prostate cancer patients. Heparinized blood (10 ml) was used to analyze the presence of galectin-3 by Western blot and ELISA.

Level of galectin-3. The association of galectin-3 secretion with the appearance of prostate cancer was examined using the human galectin-3 platinum ELISA kit (BMS279/2CE) (eBioscience).

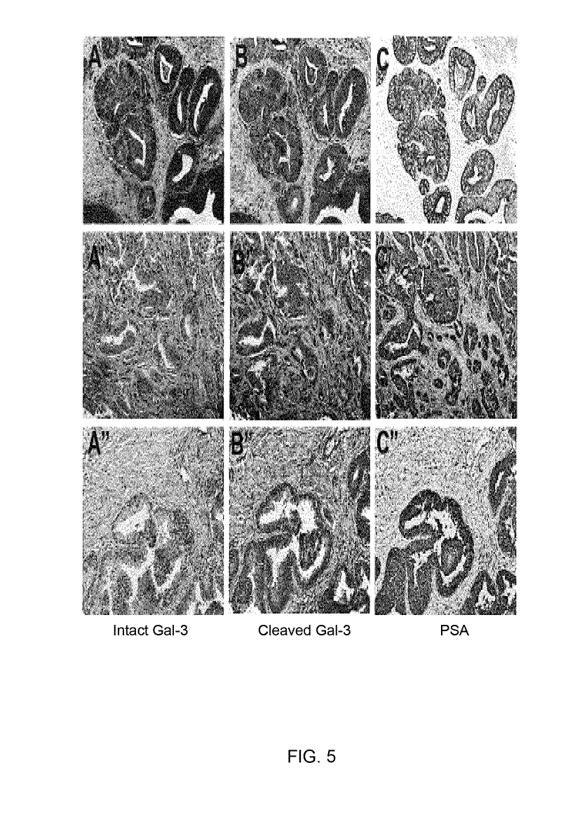

Immunohistochemical analysis. A prostate cancer tissue array was stained for intact and cleaved galectin-3 using monoclonal and polyclonal anti-galectin-3 antibodies, respectively. A prostate cancer tissue array (PR483a) from Us Biomax, Inc. (Rockville, Md.) was used. The tissue was deparaffinized, rehydrated, and microwaved for 10 min in 1 mmol/L sodium citrate buffer (pH 6.0). Endogenous peroxidase activity was blocked by 0.3% hydrogen peroxide, and nonspecific binding of immunoglobulin was minimized by blocking with Super Block (Skytek Laboratories) for 1 hr at room temperature. Sections were incubated with anti-galectin-3 antibodies (1:500 for polyclonal, 1:100 for monoclonal) overnight at 4° C., then linked with appropriate biotinylated secondary antibodies (1:500) (Vector Laboratories) and anti-PSA antibody (1:100) (Santa Cruz Biotechnology sc-7638) for 1 hr and the avidin-biotin-peroxidase complex for 30 min at room temperature, colorized by NovaRed (Vector Laboratories) for HL31 and with 3′-3′-diaminobenzidine tetrachloride with metal enhancer (cobalt) (Sigma) for TIB166 antibody. Visualization and documentation were accomplished with an Olympus BX40 microscope supporting a Sony DXC-979MD 3CCCD video camera. Two investigators evaluated results in a blinded manner. Galectin-3 immunostaining was evaluated by the percentage of positively stained epithelial cells in each section. Sections with more than 10% of positive cancer cells were regarded as positive samples. See FIG. 5.

Immunoprecipitation assays. Galectin-3 was purified using rat monoclonal TIB-166 immunoprecipitation (IP). Monoclonal rat anti-galectin-3 antibody was isolated from the supernatant of hybridoma (catalogue no. TIB-166; American Type Culture Collection). After extensive washes, galectin-3 presence in the immunoprecipitates was determined using polyclonal antibody HL31. Customized polyclonal rabbit anti-galectin-3 antibody against the recombinant whole molecule was created by Zymed Laboratories. Sera (4 ml each) from patients were incubated with 10 μg of appropriate antibody, or control rat IgG precoupled to 50 μg of protein G agarose-beads (Pharmacia) for 2 hr at 4° C. The beads were washed twice with 10 ml of lysis buffer, twice with 10 ml of lysis buffer containing 0.5 M LiCl, and twice with 10 ml of phosphate-buffered saline. Beads were boiled with 1× sample buffer and loaded on the gel for Western blot analysis.

Western blot analysis. Equal amounts of boiled sample buffer from the beads as obtained above were loaded on the gel and resolved by 10% SDS-PAGE and electroblotted onto polyvinylidene difluoride membrane (IMMOBILON® PFL, Millipore, Mass.). Membranes were quenched in a solution of TBS, containing 0.1% casein and 0.1% Tween-20 for 60 min on a rotary shaker. Blots were incubated with 1:500 diluted HL31 antibody, washed and then incubated with appropriate secondary antibodies conjugated with Alexa Fluor 680 (Invitrogen Corporation) for 30 min at room temperature. After incubation with both primary and the secondary antibodies, membranes were washed four times with TBST at 5-min. intervals. Immunoblots were visualized using the Odyssey infrared imaging system and Odyssey application software.

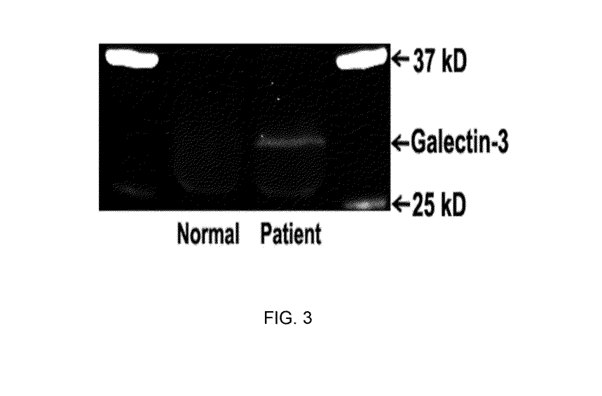

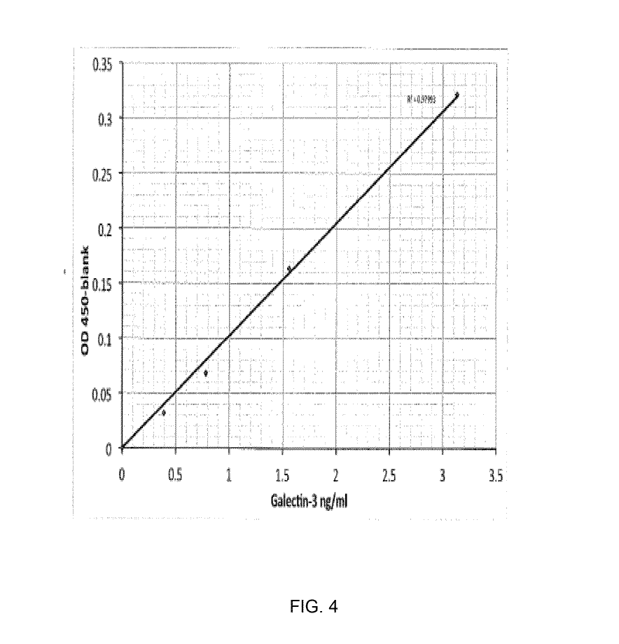

Results. Results of the Western blotting performed to determine the presence of galectin-3 in the sera used in this Example are shown in FIG. 3, in which immunostaining of blotted IPed galectin-3 with anti-galectin-3 polyclonal antibody HL31 revealed one major band with Mw˜30 kD, representing galectin-3 protein in patients with prostate cancer. The galectin-3 used to prepare the standard curve of galectin-3 ELISA was >98% pure, as judged by SDS-PAGE. The dose-response curve was linear from 390 pg/ml to 12.5 ng/ml. See FIG. 4.

As is evident from these results, the disease-free patients had a significantly lower amount of galectin-3 in the serum than did the prostate cancer patients.

Serum galectin-3 concentrations of healthy individuals and patients with prostate cancer are summarized in Table 1, which shows the galectin-3 levels of the eight healthy controls (N1-8) and eight prostate cancer patients (PC1-8). The galectin-3 levels for healthy subjects were all lower than 6 pg/ml. There was no significant difference in galectin-3 serum concentration between control samples, nor was there any correlation between serum galectin-3 levels and age or blood group (data not shown). However, the levels of galectin-3 in the sera of the eight prostate cancer patients were significantly higher than those in the control group. See Table 1. When compared to PSA concentration, the galectin-3 levels demonstrate more consistent results in predicting the risk or presence of prostate cancer than do PSA levels alone.

TABLE 1

| Sample # | galectin-3 (ng/ml) | PSA |
|---|---|---|
| PC1 | 0.41 | 619.2 |
| PC2 | 0.28 | 9.5 |
| PC3 | 0.1 | 275.1 |
| PC4 | 0.33 | 10.2 |
| PC5 | 0.32 | 0.9 |
| PC6 | 0.43 | 402.3 |
| PC7 | 0.16 | 8.2 |
| PC8 | 0.31 | 1563.1 |
| N1 | 0.06 | 1.6 |
| N2 | 0 | 1.4 |
| N3 | 0.02 | 0.56 |
| N4 | 0 | 1.1 |
| N5 | 0 | 1.1 |
| N6 | 0 | n/a |
| N7 | 0 | 0.3 |
| N8 | 0 | 0.98 |

Because alteration of galectin-3 expression in human prostate cancer can be related to detection methods, the percentage of positively stained epithelial cells in each serial section of the tissue array by HL3I and TIBI66 antibody was also examined as well as the presence and co-localization of PSA on subsequent serial section using anti-PSA antibodies, as described above.

As shown in FIG. 5 (cleavage of galectin-3 in prostate cancer and normal tissues), galectin-3 was found in both normal and cancer tissue. The amount of cleaved and intact galectin-3 in the prostate cancer tissues varied from case to case. In 43% of cancer cases, only intact galectin-3 was observed, and in 57% of prostate cancer cases there was more cleaved galectin-3 present in the tissue. See FIG. 5, A-C and A′-C′. In normal tissue, however, more cleaved galectin-3 was observed than intact galectin-3. See FIG. 5, A″-C″. This data further suggests a dysregulation of galectin-3 cleavage in cancerous prostate tissue, compared to that of normal tissue.

### Example 3

A clinical trial is conducted in five groups of patients older than 18 years of age, with the five groups as follows: Group 1: Control group, men with no history of current invasive cancer; Group 2: newly diagnosed patients with intact prostate cancer; Group 3: patients who have no evidence of disease recurrence post local therapy; Group 4: patients who have rising PSA after local therapy (defined as any value above undetectable); and Group 5: patients with metastatic prostate cancer (castrate-sensitive and/or castrate-resistant).

Two 5 cc gold top tubes of blood samples will be collected from males in each group. Blood cells will be removed from the samples and serum will be saved at −20° C. until all samples are collected. The most recent historical PSA level in the subject's medical record is also collected.

Level of galectin-3. Heparinized blood is used to analyze the presence of galectin-3 in two replicates by Western blot and ELISA. The level of galectin-3 is detected using a human galectin-3 platinum ELISA kit (BMS279/2CE) (eBioscience) For each study participant, the mean of the two replicates is used in statistical analysis.

Immunohistochemical analysis. A PCa tissue array (PR483a) is used from US Biomax, Inc. It is deparaffinized, rehydrated, and microwaved for 10 min in 1 mmol/L sodium citrate buffer (pH 6.0). Endogenous peroxidase activity is blocked by 0.3% hydrogen peroxide, and nonspecific binding of immunoglobulin is minimized by blocking with Super Block (Skytek Laboratories) for 1 hr at room temperature. Sections of PCa tissue array (PR483a) are incubated with anti-galectin-3 antibodies (1:500 for polyclonal, 1:100 for monoclonal) overnight at 4° C. The tissue arrays are incubated with appropriate biotinylated secondary antibodies (1:500; Vector Laboratories) and anti-PSA antibody (1:100, Santa Cruz Biotechnology) for 1 hr and the avidin-biotin peroxidase complex for 30 min at room temperature. The sections are colorized by NovaRed (Vector Laboratories) for HL31 and with 3′-3′-diaminobenzidine tetrachloride with metal enhancer (cobalt) (Sigma) for TIBI66 antibody. Visualization and documentation are accomplished with an Olympus BX40 microscope supporting a Sony DXC-979MD 3CCCD video camera. Two investigators evaluate the laboratory results without knowledge of patient group membership. Galectin-3 immunostaining is evaluated by the percentage of positively stained epithelial cells in each section of PCa tissue array (PR483a). Sections with more than 10% of positive cancer cells are regarded as positive samples.

Immunoprecipitation assays. Galectin-3 is purified using rat monoclonal TIB-166 immunoprecipitation (IP). Monoclonal rat anti-galectin-3 antibody is obtained from the supernatant of hybridoma (catalogue no. TIB-166; American Type Culture Collection). After extensive washes, galectin-3 presence in the immunoprecipitates is determined using polyclonal antibody HL31. Customized polyclonal rabbit anti-galectin-3 antibody against the recombinant whole molecule (Zymed Laboratories) is used in the assay. Four milliliters of serum from patients are incubated with 10 pg of appropriative antibody, or control rat IgG precoupled to 50 μl of protein G agarose-beads (Pharmacia) for 2 hr at 4° C. The beads are washed twice with 10 ml of lysis buffer, twice with 10 ml of lysis buffer containing 0.5 M LiCl, and twice with 10 ml of phosphate-buffered saline. Beads are boiled with lx sample buffer and loaded on the gel.

Western blot analysis. Equal amounts of boiled sample buffer from the beads is loaded on the gel and resolved by 10% SDS-PAGE and electroblotted onto polyvinylidene difluoride membrane (Immobilon PFL). Membranes are quenched in a solution of TBS, containing 0.1% casein and 0.1% Tween-20 for 60 min on a rotary shaker. Blots are incubated with 1:500 diluted HL31 antibody, washed and then incubated with appropriate secondary antibodies conjugated with Alexa Fluor 680 (Invitrogen Corporation) for 30 min at room temperature. After incubation with both primary and the secondary antibodies, membranes are washed four times with TBST (TBS, containing 0.1% Tween-20) at 5-min intervals. Immunoblots are visualized using the Odyssey infrared imaging system and Odyssey application software (LI-COR Biosciences).

Statistics. The primary statistical endpoint is the mean level of serum Gal-3. Within each group, the mean Gal-3 level is estimated to within 0.40 standard deviations (SDs) of the true mean, with 90% confidence. This study design requires N=19 men per group. Separately for each group of study subjects, the Gal-3 data is summarized with standard descriptive statistics (N, median, interquartile range (IQR), mean, SD, minimum, maximum, and the 90% confidence interval (CI) for the mean). Statistical graphics (boxplots, dotplots) of the Gal-3 data is also be generated for each group. Separately for each group of study subjects, the Pearson correlation between serum Gal-3 and serum PSA will be calculated, along with its 90% Cl.

The results will further confirm the utility of the claimed methods and kits.

Phrases such as “significantly different”; “significantly elevated” and other similar phrases should be interpreted to mean an observed difference or increased level that is greater than what would be expected to occur by chance alone. A difference or elevation described herein is significantly different or significantly elevated if the p-value of a statistical test is equal to or less than 0.5.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in the ability to detect the presence of prostate cancer.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, where references have been made to patents and printed publications throughout this specification, each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1.-54. (canceled)

55. A kit for improving the reliability of prostate specific antigen (PSA)-based prostate cancer progression testing comprising at least one galectin-3 detection assay and at least one PSA detection assay.

56. A kit of claim 55, further comprising instructions for performing the detection assays and instructions for determining whether the subject has progressed prostate cancer.

57. A kit of claim 56, wherein the instructions comprise reference levels.

58. A kit of claim 56, further comprising reference samples for determining reference levels of galectin-3 and PSA.

59. A method for monitoring the progression of prostate cancer in a subject comprising:
determining a galectin-3 level and a prostate specific antigen (PSA) level in a biological sample obtained from the subject;
comparing the measured galectin-3 level with a galectin-3 reference level;
comparing the measured PSA level with a PSA reference level; and
determining that the prostate cancer has progressed if the level of galectin-3 in the biological sample is significantly elevated compared to the galectin-3 reference level and the level of PSA in the biological sample is significantly elevated compared to the PSA reference level;
wherein the method is more reliable at monitoring the progression of prostate cancer than a method that utilizes a prostate specific antigen (PSA) test alone.

60. A method of claim 59, wherein the galectin-3 reference level and/or PSA reference level are individually derived from (i) an individual who does not have prostate cancer, (ii) a group of individuals who do not have prostate cancer; (iii) the subject before diagnosis with prostate cancer; or (iv) the subject at the beginning of a treatment regimen for prostate cancer.

61. A method of claim 59, wherein the biological sample is a blood sample, a plasma sample, or a serum sample.

62. A method for improving the reliability of PSA-based prostate cancer progression testing comprising:
determining a galectin-3 level and a prostate specific antigen (PSA) level in a biological sample obtained from the subject;
comparing the measured galectin-3 level with a galectin-3 reference level;
comparing the measured PSA level with a PSA reference level; and
determining that the prostate cancer has progressed if the level of galectin-3 in the biological sample is significantly elevated compared to the galectin-3 reference level and the level of PSA in the biological sample is significantly elevated compared to the PSA reference level;
wherein the reliability of the method for prostate cancer progression testing is more reliable than a method that utilizes a prostate specific antigen (PSA) test alone.

63. A method of claim 62, wherein the galectin-3 reference level and/or PSA reference level are individually derived from (i) an individual who does not have prostate cancer, (ii) a group of individuals who do not have prostate cancer; (iii) the subject before diagnosis with prostate cancer; or (iv) the subject at the beginning of a treatment regimen for prostate cancer.

64. A method of claim 62, wherein the biological sample is a blood sample, a plasma sample, or a serum sample.


SEQUENCE LISTING (4 sequences)

SEQ ID NO: 1 - protein, 250 aa, Homo sapiens [sequence omitted]

SEQ ID NO: 2 - protein, 107 aa, Homo sapiens
MADNFSLHDALSGSGNPNPQGWPGAWGNQPAGAGGYPGASYPGAYPGQAPPGAYPGQAPPGAYPGAPGAYPGAPAPGVYPGPPSGPGAYPSSGQPSATGAYPATGPY

SEQ ID NO: 3 - protein, 143 aa, Homo sapiens
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 4 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP